(19) United States
(12) Patent Application Publication
(10) Pub. No.: US 2026/0335403 A1
(43) Pub. Date: Oct. 29, 2026

(54) ENDOGENOUS CAPSIDS AS PRO-FERTILITY FACTORS

(71) Applicant: The Trustees of Columbia University in the City of New York, New York, NY (US)

(72) Inventors: Luke Berchowitz, New York, NY (US); Thomas Wood, New York, NY (US); Harrison Cullen, New York, NY (US)

(21) Appl. No.: 19/653,898

(22) Filed: Apr. 21, 2026

Related U.S. Application Data
(63) Continuation of application No. PCT/US2024/053329, filed on Oct. 29, 2024.
(60) Provisional application No. 63/546,397, filed on Oct. 30, 2023.

(51) Int. Cl. A61K 38/17; A61P 15/08; C07K 1/14; C07K 14/47

(52) U.S. Cl. CPC A61K 38/1709; A61P 15/08; C07K 1/14; C07K 14/47

(57) ABSTRACT

Methods and compositions for improving fertility or treating age-related infertility.

### CROSS-REFERENCE TO RELATED APPLICATIONS

This application is a continuation of PCT International Application No. PCT/US2024/053329, filed Oct. 29, 2024, which claims benefit of U.S. Provisional Application No. 63/546,397, filed Oct. 30, 2023, the contents of which are hereby incorporated by reference.

### BACKGROUND OF THE INVENTION

The disclosures of all publications, patents, patent application publications and books referred to in this application are hereby incorporated by reference in their entirety into the subject application to more fully describe the art to which the subject invention pertains.

The median age of new parents in the US has risen dramatically over the past 50 years and, consequently, the need to understand the genetic factors underlying age-dependent subfertility are more urgent than ever. We have identified two human genes important for safeguarding male and female reproductive health with age that evolved from parasitic genetic elements called retrotransposons. The reproductive roles of these genes are unknown. Further understanding the fertility-related processes that they govern and determining their molecular mechanisms of action is desirable.

### SUMMARY OF THE INVENTION

Herein is disclosed that PNMA1 and PNMA4 form capsids in testis and/or ovaries and such capsids can be used as a therapeutic in relation to age-based subfertility.

A method for improving fertility or treating infertility in a subject comprising administering to the subject an amount of capsids comprising PNMA1 and/or PNMA4 effective to improve fertility or treat infertility in a subject.

A pharmaceutical composition comprising an amount of purified capsids comprising PNMA1 and/or PNMA4 and a pharmaceutically acceptable carrier.

A method of making a pharmaceutical composition for treating infertility or subfertility in a subject comprising isolating from a biological sample capsids comprising PNMA1 and/or PNMA4 and admixing the capsids with a pharmaceutically acceptable carrier.

### BRIEF DESCRIPTION OF THE FIGURES

FIG. 1: Yeast Ty3 elements are activated by host meiotic factors and repressed by Rim4 condensates. TF = transcription factor

FIGS. 2A-2C: Thousands of gag-like capsid genes in the human genome originate from four clades, but only Metaviridae-derived capsid genes are retained across placental mammals. (A) Maximum-likelihood phylogenetic tree of 491 full-length capsid genes in the human genome. Positive control sequences (black) from LTR retroelement capsids cluster with their expected viral class (HIV, dARC1, and Ty3). (B) Seventeen fill-length capsid genes derived from Metaviridae retrotransposons are conserved in placental mammals. *In rodents, gene conversion led to the replacement of ZCCHC18 with a second copy of ZCCHC12. **PNMA6E/F appear as identical duplicated in many mammalian genomes. (C) Most gag-like capsid genes are robustly expressed in male and female reproductive tissues (data from GTEx project, gtexportal.org).

FIG. 3. Mammalian gag-like genes PNMA1 and PNMA4 are activated by meiotic transcription factors and bound by Rim4 analog DAZL.

FIGS. 4A-4B: M. musculus Pnma1 (A) and Pnma4 (B) loci highlighting features and deleted regions. Pnma1 and Pnma4 deletions were generated by CRISPR/Cas9 genome editing. Note that we needed to delete a larger region flanking the Pnma4 genedue to sgRNA uniqueness restrictions. CRISPR reagents (sgRNA target sites shown in dashed lines) were injected into fertilized mouse eggs and successful deletion was assessed by sequencing. Shown are chromosomal coordinates, annotated transcripts (CDS in dark blue, UTRs in light blue, and introns in white), deleted regions, and sequencing reads (below, maroon) confirming deletion of Pnma1 and Pnma4.

FIGS. 5A-5D: Pnma mutant mice exhibit a subfertility phenotype. (A) Number of pups produced by breeding pairs of wild type CF-1 female testers and control (n = 40), Pnma1 ^{-/-} (n = 40), or Pnma4 ^{-/-} (n = 28) males or (B) wild type B6D2F1/J males paired with control (n = 20), Pnma1 ^{-/-} (n = 20), or Pnma4 ^{-/-} (n = 14) females. Shown are the results of pairings over the course of 2-5.5 months. Graphs indicate mean +/- SEM, ** = p < 0.005. (C, D) Total zero pup litters accumulated over the course of the fertility trials in (A, B).

FIGS. 6A-6D: Testicular defects underlie subfertility of Pnma mutant males. (A) Testes from control (n = 52, pooled wild type and hets), Pnma1 -/- (n = 54), and Pnma4 ^{-/-} (n = 25) males (1.5 - 6 months) were weighed. (B) Cauda epididymis was dissected and sperm were counted at the indicated age. (C) Serum testosterone was measured in males at the indicated age. Box and whisker plots indicate median, min/max, and first/third quartiles. Graphs indicate mean +/- SEM. Results of 2-tailed t-tests are indicated (* = p <0.05; ** = p <0.005) D) PAS-stained tubule sections (~ 3 months).

FIGS. 7A-7D: Ovarian defects underlie subfertility phenotypes in Pnma mutant females. (A) Ovaries from control (n = 24, pooled wild type and hets). Pnma1 ^{-/-} (n = 38), and Pnma4 ^{-/-} (n = 13) females (1.5 – 6 months) were weighed. (B, C) Antral follicles and follicular cysts per mm2 were measured from PAS-stained ovary sections at the indicated ages. Graphs indicate mean +/- SEM. Results of 2-tailed t-tests are indicated (* p = <0.05 compared to control) D) PAS-stained ovary sections (~ 3 months). Key features are indicated by arrows.

FIGS. 8A-8B Pnma mutant mice become obese with age. (A) Body weights of control (pooled wild type and het), Pnma1 ^{-/-}, and Pnma4 ^{-/-} were measured at the indicated ages. Graphs indicate mean +/- SEM. Results of 2-tailed t-tests are indicated (* = p <0.05) (B) Imaged of indicative abdominal morphology for dissected ~6 month mice.

FIGS. 9A-9D: PNMA proteins have the capacity to form capsids that exit human cells. (A - D) Plasmids expressing V5-tagged PNMA4 or ARC (positive control) were transfected into HEK-293T cells. (A) Culture medium of transfected cells was collected, filtered, concentrated over a 100 kD molecular weight cutoff (MWCO) filter, and fractionated by size exclusion chromatography. Fractions were analyzed by immunoblot. (B) Sample transmission electron micrograph or PNMA multimeric assemblies in vitreous ice. (C) 2D class averages from a data collection. (D) Preliminary reconstruction of a PNMA4 capsid.

FIGS. 10A-10B: PNMA1 and PNMA4 are expressed in human ovaries. (A) Analysis of uniquely mapping single-cell RNAseq reads for PNMA1-5 loci expressed in MII human oocytes (data from Yuan et al., 2021 and Zhang et al., 2018)^{38, 39}. (B) Human ovaries from young (23-29 years of age, N = 4) and older (49-54 years of age, N = 4) donors were analyzed by single-nuclei RNAseq. Uniquely mapping reads for PNMA1 and PNMA4 loci were assigned to ovarian tissue types based on clustering analysis. Note that the majority of values = 0 and lie underneath the x axis (error bars represent SEM). Total n of each cell type and percentage of positive cells is indicated above the plots. Statistical significance was determined by Mann-Whitney test (n.s. p > 0.05, * p ≤ 0.05, ** p ≤ 0.01, *** p ≤ 0.001, **** p ≤ 0.0001).

FIGS. 11A-11B: PNMA1 and PNMA4 are conserved among eutherians. (A, B) Architecture of the human PNMA1 and PNMA4. Loci are colored according to domains: capsid domain (CA, magenta), linker (L, lilac), and RNA-binding domain (RBD, purple). The promoter (green) and transcription start sites are shown using arrows. A black triangle on the phylogenetic tree (left) indicates the point of the first expansion of the ancestral PNMA locus leading to PNMA1-5. PNMA1 is universally retained across placental mammals as an intact gene, whereas PNMA4 has experienced lineage-specific pseudogenization (boxed x’s). Conservation at each amino acid is shown with a vertical black line for fifteen eutherian mammals, three marsupials, and two vertebrate outgroups. The histone modification for active transcription (H3K27Ac) in humans is shown along the bottom.

FIGS. 12A-12I: Male mice lacking Pnma1 or Pnma4 prematurely lose reproductive capacity. (A) Wild type male control mice (gray), Pnma1 -/- (cyan), or Pnma4 -/- (purple) were crossed biweekly to CF-1 female fertility tester mice (N = 5 pairs for each genotype-timepoint combination). Pup numbers for each cross were recorded. (B, C) Testes (n above graph) from control (pooled wild type and heterozygous, gray), Pnma1 -/- (cyan), Pnma4 -/- (purple), or Pnma1 -/- Pnma4 -/-double mutant (red) mice were weighed. Representative images and quantifications are shown. (D) Sperm counts from dissected cauda epididymides. The number of individuals (N) for each genotype and age are shown above. (E) Serum testosterone (from N individual males) at the indicated ages.(F) PAS-stained tubule sections from 6-month-old mouse testes; devoid tubules are indicated by yellow arrowheads.(G) Devoid tubules (from n testes) as percentage of total. (H) Sections from 12-month-old testes were analyzed by TUNEL and DAPI staining. TUNEL-positive cells are indicated by yellow arrowheads. (I) Percent TUNEL-positive cells per tubule (n indicated by genotype to the right).Statistical significance was determined by one-way ANOVA with correction for multiple comparisons and student’s t-test (n.s. p > 0.05, * p ≤ 0.05, ** p ≤ 0.01, *** p ≤ 0.001, **** p ≤ 0.0001). Error bars indicate SEM.

FIGS. 13A-13J: Female mice lacking Pnma1 or Pnma4 have age-dependent reproductive defects. (A) Wild type female wild type mice (gray), Pnma1 -/- (cyan), or Pnma4 -/- (purple) were crossed to B6D21/J male fertility tester mice (N = 5 pairs for each genotype-timepoint combination). Pup numbers for each cross are plotted over time. (B) PAS-stained ovarian sections from 3-month-old mouse ovaries. Black arrows denote antral follicles, yellow arrows denote abnormal follicles, and green arrows denote follicular cysts. (C) Representative images of ovaries from the four genotypes. (D) Ovaries (n above graph) from control (pooled wild type and heterozygous, gray), Pnma1 -/- (cyan), Pnma4 -/- (purple), or Pnma1 -/- Pnma4 -/-double mutant (red) mice were weighed. (E) The number of antral follicles at the indicated ages per mm². The number of individuals (N) for each genotype and age are shown. (F) The number of follicular cysts per mm² by age and genotype (N’s in graph). (G) Germinal vesicle (GV) oocytes were collected from six-month-old females and meiotically induced. Meiotic progression was analyzed by tubulin immunofluorescence. Percentage of meiosis II oocytes was recorded. (H) GV oocytes were collected from seven-month-old wild-type and double mutant mouse ovaries. We injected mRNA encoding mClover-MAP4 (microtubule-binding protein) and H2B-mScarlet (histone) and imaged oocytes live for ~18 hours, recording (I) the time from nuclear envelope breakdown (NEBD) to anaphase I and (J) lagging chromosome percentages. Statistical significance was determined by one-way ANOVA with correction for multiple comparisons and student’s t-test (n.s. p > 0.05, * p ≤ 0.05, ** p ≤ 0.01, *** p ≤ 0.001, **** p ≤ 0.0001). Error bars indicate SEM.

FIGS. 14A-14C: Pnma1 and Pnma4 mutants gain abdominal fat with age. (A) Body weights (N above graph) of control (pooled wild type and heterozygote, gray), Pnma1 -/- (cyan), Pnma4 -/- (purple), or Pnma1 -/- Pnma4 -/-double mutant (red) at the indicated ages. (B) Representative images of abdominal fat at six months in each genotype. (C) Food intake of 3-month wild type, Pnma1 -/-, Pnma4 -/-, or Pnma1 -/- Pnma4 -/-double mutant mice (N = 10 mice) was monitored daily for 7 days. Statistical significance was determined by one-way ANOVA with correction for multiple comparisons (n.s. p > 0.05, * p ≤ 0.05, ** p ≤ 0.01, *** p ≤ 0.001, **** p ≤ 0.0001). Error bars indicate SEM.

FIG. 15A-15G: PNMA proteins form capsid-like structures that can exit human cells. (A, B) Top panels depict TEM micrographs of negatively stained recombinant PNMA1 (A) and PNMA4 (B) protein. Bottom panels depict 2D class averages from cryoSPARC. (C) Experimental setup: 3V5-tagged PNMA1, PNMA4, and ARC (control exported capsid) expression plasmids were transfected into HEK-293T cells. mCherry (control non-capsid) was co-expressed from the transfected plasmid. Culture medium was collected, spun, and filtered to remove cells and debris. Filtrate was fractionated over a 20% sucrose cushion to enrich for capsids, which fractionate as large particles at the bottom of the cushion (pellet). Sucrose, meniscus, spun media, and pellet fractions were collected. Unfractionated media input and cell lysate samples were also collected. (D) PNMA, PNMA4, ARC (anti-V5), and mCherry protein levels by immunoblot. Untransfected controls (control input) are at the far right of each blot. Asterisks (*) mark the cross-reacting BSA band (abundant in growth medium). Note the presence of BSA in the untransfected controls and absence of BSA in pellet/lysate. (E) Experimental setup: Lysate was prepared from ten testes from wild type C57BL6/J mice were collected at 3 months. Lysate was fractionated by velocity step gradient (38,000 rpm, 3 hours) over a double sucrose cushion (25% and 70%). Capsid-like structures migrate to the interface between 25%-70% sucrose (70% meniscus). The 70% meniscus was further fractionated by isopycnic (equilibrium) centrifugation on an iodixanol step gradient; the photograph shows the centrifuge tube after this step. (F-G) PNMA4, MLV p30 gag, and GAPDH protein levels were determined by immunoblot in each fraction at the end of the double sucrose fractionation and (G) iodixanol fractionation.

FIG. 16: Model for PNMA1 and PNMA4 function.

### DETAILED DESCRIPTION OF THE INVENTION

A method for improving fertility or treating infertility in a subject comprising administering to the subject an amount of capsids comprising PNMA1 and/or PNMA4 effective to improve fertility or treat infertility in a subject.

In embodiments, the capsids have been previously obtained from a mammalian subject.

In embodiments, the capsids have been previously obtained mammalian testis or ovary tissues.

In embodiments, the PNMA1 and/or PNMA4-comprising capsids are obtained from cultured cells bioengineered to express PNMA1 and/or PNMA4.

In embodiments, the capsids are endogenous capsids.

In embodiments, the capsids have not been genetically engineered.

In embodiments, the capsids have not been altered to contain RNA.

In embodiments, the capsids are administered directly to the testes of a male subject or to the ovary of a female subject.

In embodiments, the capsids have been purified from a biological sample by size and/or by density.

In embodiments, the capsids have been purified from a biological sample via antibody purification.

In embodiments, the capsids have been purified from a biological sample which is a reproductive tissue.

In embodiments, the capsids are human.

In embodiments, the administration of the capsids effects an increase in testosterone in the subject.

In embodiments, the administration of the capsids effects an increase in estrogen in the subject.

In embodiments, the subject has an age-related fertility decline or sub-fertility.

In embodiments, the capsids comprise PNMA4 but not PNMA1.

In embodiments, the subject is a human.

In embodiments the methods further comprise initially identifying the patient has having an infertility or a subfertility prior to administering the amount of capsids.

A pharmaceutical composition comprising an amount of purified capsids comprising PNMA1 and/or PNMA4 and a pharmaceutically acceptable carrier.

In embodiments, the capsids comprise PNMA4 but not PNMA1.

In embodiments, the capsids have been previously obtained mammalian testis or ovary tissues.

A method of making a pharmaceutical composition for treating infertility or subfertility in a subject comprising isolating from a biological sample capsids comprising PNMA1 and/or PNMA4 and admixing the capsids with a pharmaceutically acceptable carrier.

In embodiments, the method further comprises purifying the capsids comprising PNMA1 and/or PNMA4 prior to admixing the capsids with a pharmaceutically acceptable carrier.

In embodiments, the method comprises purifying capsids comprising PNMA4 but not PNMA1, and the resultant pharmaceutical composition contains capsids comprising PNMA4 but not PNMA1.

In embodiments the sequence of PNMA4 is set forth in Genbank in AAG31786.1, BAB14788.1, BAG37903.1, EAW81516.1, or EAW81516.1. In embodiments the sequence of PNMA1 is set forth in Genbank in CABD030093180.1.

Administration in an embodiment of the methods is intravenous. Administration in an embodiment of the methods is via infusion. Administration can also be auricular, buccal, conjunctival, cutaneous, subcutaneous, endocervical, endosinusial, endotracheal, enteral, epidural, via hemodialysis, interstitial, intrabdominal, intraamniotic, intra-arterial, intra-articular, intrabiliary, intrabronchial, intrabursal, intracardiac, intracartilaginous, intracaudal, intracavernous, intracavitary, intracerebral, intracisternal, intracorneal, intracoronary, intradermal, intradiscal, intraductal, intraepidermal, intraesophagus, intragastric, intravaginal, intragingival, intraileal, intraluminal, intralesional, intralymphatic, intramedullary, intrameningeal, intramuscular, intraocular, intraovarian, intraepicardial, intraperitoneal, intrapleural, intraprostatic, intrapulmonary, intrasinal, intraspinal, intrasynovial, intratendinous, intratesticular, intrathecal, intrathoracic, intratubular, intratumor, intratympanic, intrauterine, intravascular, intraventricular, intravesical, intravitreal, laryngeal, nasal, nasogastric, ophthalmic, oral, oropharyngeal, parenteral, percutaneous, periarticular, peridural, rectal, inhalationally, retrobulbar, subarachnoid, subconjuctival, sublingual, submucosal, topically, transdermal, transmucosal, transplacental, transtracheal, ureteral, uretheral, and vaginal.

In embodiments, the capsids are present in 0.1 to 10 micrograms of purified capsid per 10 microliters of carrier e.g. for injection. In embodiments, the capsids are present in 0.5 to 5 micrograms of purified capsid per 10 microliters of carrier. In embodiments, the capsids are present in 1 to 2 micrograms of purified capsid per 10 microliters of carrier.

In embodiments, the capsids are present at a 1 to 500 nanomolar concentration. In embodiments, the capsids are present at a 10 to 250 nanomolar concentration. In embodiments, the capsids are present at a 15 to 50 nanomolar concentration. In embodiments, the capsids are present at a 20-40 nanomolar concentration. Administration can be into reproductive organ, for example, a teste or an ovary.

"Pharmaceutically acceptable carrier" means a carrier that is useful in preparing a pharmaceutical composition that is generally safe, non-toxic and neither biologically nor otherwise undesirable, and includes a carrier that is acceptable for veterinary use as well as human pharmaceutical use. "A pharmaceutically acceptable carrier" as used in the specification and claims includes both one and more than one such carrier. Suitable pharmaceutically acceptable carriers are well known in the art.

“And/or” as used herein, for example with option A and/or option B, encompasses the separate embodiments of (i) option A, (ii) option B, and (iii) option A plus option B.

All combinations of the various elements described herein are within the scope of the invention unless otherwise indicated herein or otherwise clearly contradicted by context.

This invention will be better understood from the Experimental Details, which follow. However, one skilled in the art will readily appreciate that the specific methods and results discussed are merely illustrative of the invention as described more fully in the claims that follow thereafter.

### EXPERIMENTAL - I

Subfertility arises from complex interacting variables such as age, environment, and genetics. As the median age of new parents and prevalence of subfertility continues to rise, the genetic factors underlying subfertility represent an unknown of increasing importance. A key to understanding these factors could be hidden in the ‘dark matter’ of the human genome derived from mobile elements called retrotransposons. While these parasitic sequences proliferate via an RNA-based ‘copy and paste’ mechanism, inactivated elements can serve as a genetic reservoir for the evolution of new genes. We discovered that meiotic expression of yeast Metaviridae retrotransposons requires an essential meiotic transcription factor. We wondered if similar phenomena existed in humans. While Metaviridae elements no longer retain retrotransposition capacity in mammals, our genomes are littered with their mutated remnants, some of which are now useful genes. Our phylogenetic analysis revealed seventeen human Metaviridae-derived genes conserved among placental mammals. Fourteen are expressed in testis and/or ovary, and at least two — PNMA1 and PNMA4 — are regulated by master germ cell transcription factors Stra8 and Mybl1. Furthermore, transcripts of both genes are bound by the essential RNA-binding protein DAZL in human ovaries and mouse testes. Clearly these genes are developmentally regulated in developing germ cells. But are they performing vital reproductive functions?

To investigate this question, we generated two mouse models harboring deletions of Pnma1 or Pnma4. Male and female mice lacking either of these genes exhibit age-dependent subfertility and corresponding abnormal gonadal morphology. Our data strongly support the notion that PNMA1 and PNMA4 are providing a functional benefit in testis and ovary, and we propose to determine their unknown roles in these tissues. PNMA-family genes evolved from gag sequences. Retrotransposon gag proteins have two primary functions: bind RNA cargos and package these payloads in capsids. When we expressed PNMA1 and PNMA4 in human cells, we found that they form capsid-like structures that escape the cell into the growth medium. These results have led us to the surprising hypothesis that mammalian reproductive tissues are expressing capsid proteins that promote testicular and ovarian health via transfer of a molecular cargo.

### Identify the fertility-associated processes mediated by PNMA1 and PNMA4

To understand how PNMA1 and PNMA4 promote fertility, clarification of the reproductive processes that are disrupted in mice lacking these genes will be informative. Because our analysis suggests that male and female mutant mice exhibit premature reproductive aging, we will conduct a time course analysis of all phenotypes. We will perform fertility trials in single, double knockout, and control mice over six months. In mutant males, we will analyze testis morphology, sperm count, and serum hormone levels. In mutant females, we will analyze ovarian morphology focusing on follicle development, ovarian reserve, and cyst formation. We will also measure serum peptide and steroidal hormone levels. Because Pnma1/ 4 exhibit peak expression during pachynema, we will monitor key meiotic processes in mutants by staining developing spermatocyte and oocyte nuclei for instructive cytological and histological meiotic markers. In parallel, to circumvent challenges of studying female meiosis in mice, we will analyze meiotic chromosome segregation and spindle morphology in live-imaged oocytes collected from mutant and control animals.

### Dissect the molecular mechanisms underlying PNMA1 and PNMA4 function

To determine the molecular mechanisms underlying PNMA1 and PNMA4 function, we will assess their subcellular and temporal expression patterns in developing mouse testis and ovary. Because gag proteins typically bind nucleic acids, we will conduct nucleic acid labelling followed by ChIP-seq and/or HITS-CLIP to determine whether PNMA1 and/or PNMA4 are performing DNA and RNA-binding functions. We will use IP-mass spectrometry to determine protein co-factors. These approaches will allow us to identify the targets and binding partners for these two proteins and will provide important insight into how gag proteins have evolved to identify and regulate nucleic acid sequences in germ cells. Lastly, we will determine whether the pro-apoptotic functions of PNMA1 and PNMA4 are important for germ cell quality control.

### Determine the structure and function of PNMA capsids

PNMA proteins assemble into capsid-like structures which we hypothesize are important for function. To test this hypothesis, we will purify capsids from mouse reproductive tissues using density gradient fractionation and size exclusion chromatography (with established positive controls) and determine their cargo by sequencing and mass spectrometry. High-resolution structures determined using single particle cryo-EM will provide insight into how capsids selectively package and deliver molecular cargos that are critical for fertility.

Globally and in the US, the median age of new parents and the prevalence of subfertility has risen dramatically over the past 50 years. As an increasing percentage of the population wanting to have children confronts age-based fertility decline, the need to understand the genetic factors underlying subfertility is becoming more urgent. We have identified conserved mammalian genes, evolved from parasitic genetic elements called retrotransposons, that are robustly expressed in male germ cells and ovaries. We hypothesize that these genes play important roles in germ cell development and fertility maintenance, which is strongly supported by our data.

### Retrotransposon sequences are primed to develop functions that benefit developing germ cells

Retrotransposons are a class of selfish mobile (i.e., transposable) elements that propagate through an RNA intermediate. This ‘copy and paste’ proliferation mechanism preserves the original element while generating DNA copies that integrate at other loci. The success of these elements is evident in the human genome, of which over 45% is composed of retrotransposons and their mutated remnants. While retrotransposon-derived sequences pose a significant burden to host genomes, they correspondingly present opportunities for evolutionary innovation as a feedstock for the evolution of new genes by a process termed “domestication”.

To understand how retrotransposons may become useful genes, it is important to consider the imperatives of their life cycle: they must proliferate in germ cells (or their stem cell precursors) to avoid extinction and maintain vertical inheritance. The process of domesticating formerly parasitic elements could manifest in any cell type, but we hypothesize that neofunctionalization will frequently be associated with reproductive biology functions based on the replication niche (i.e., developing germ cells) of their parasitic ancestors.

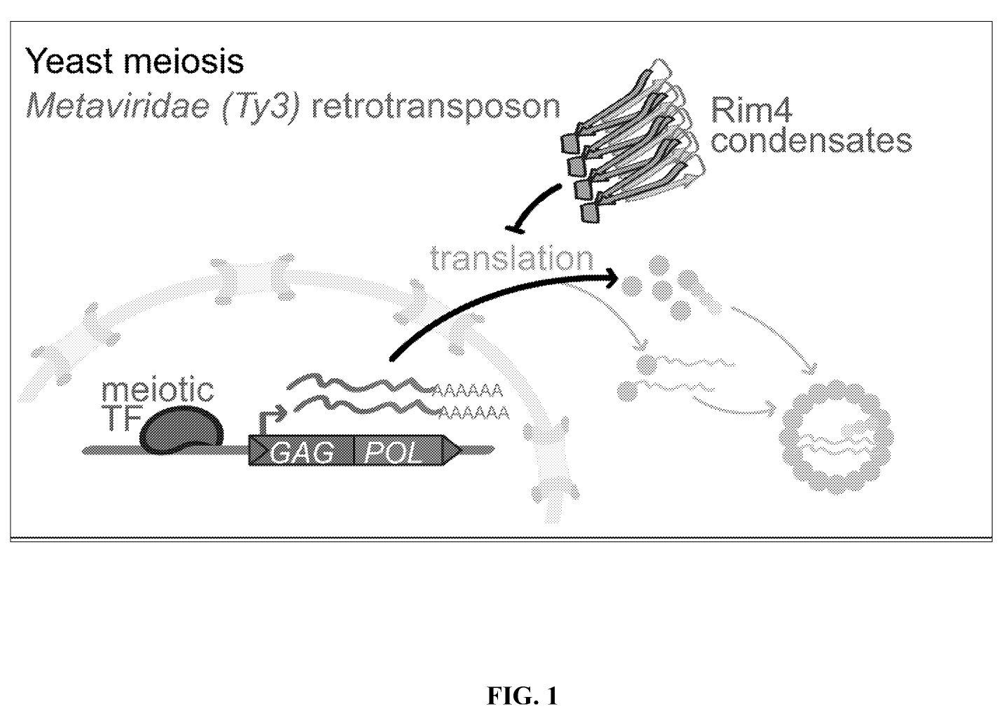

This work is rooted in our long-term interest in understanding how genetic information is passed through generations and how genome integrity is maintained during germ cell development. We previously discovered that yeast Metaviridae (Ty3) retrotransposons co-opt binding sites of an essential meiotic transcription factor upstream of their integration site, thereby linking theiractivation to gametogenesis. We also identified an opposing mechanism by which meiotic cells defend their genome against retrotransposons. We found that developing yeast gametes use condensates of the RNA-binding protein Rim4 to repress the translation of retrotransposon mRNA, thereby halting the proliferation cycle(FIG. 1). The next step (and the beginning of the research proposed here) was to determine whether similar phenomena exist in humans.

Metaviridae no longer retain retrotransposition capacity in mammals. The human genome is, however, littered with sequences derived from inactive Metaviridae elements, some of which are now domesticated genes⁴. Are these genes, like their parasitic ancestors, expressed in developing germ cells? If so, have they evolved to play important roles in germ cell development? We reasoned that candidate human Metaviridae-derived genes with the potential to benefit developing germ cells will fulfill the following criteria: a) broad conservation among mammals and b) robust expression in reproductive tissues. This idea led to a fortuitous collaboration between the Berchowitz and Wiedenheft labs, the latter of whom were elucidating the evolutionary trajectories of retrotransposon-derived genes across ~150 million years of mammalian evolution.

While total infertility (i.e., sterility) adversely affects numerous people wanting to have children, subfertility, defined as any form of reduced fertility with prolonged time of unwanted non-conception, represents an equally pressing health challenge due, in part, to its prevalence. Total infertility is an unambiguous condition — its etiology can often be pinned to a discrete cause such as a genetic mutation or the side effect of a drug or medical procedure. Subfertility, on the other hand, often has a complex basis resulting from the combined effects of many interacting factors such as genetics, aging, and environment. Although the concept of a fertility cliff is (rightfully) becoming outdated, the fact remains that subfertility increases with age in both males and females, regardless of socioeconomic status. Our understanding of the genetic factors underlying subfertility remains murky, especially when compared to knowledge of total infertility where genetic abnormalities account for approximately 50% of cases. Our data (discussed below) provide a basis for the hypothesis that two retrotransposon-derived capsid genes are important for fertility maintenance with age, however, their loss does not cause total infertility. We propose that understanding the reproductive biology governed by capsid genes could lead to crucial advances in our understanding of the genetic bases underlying subfertility.

Genes that originated from active retrotransposons or the remains of inactivated elements are often robustly expressed in reproductive tissues; notably in testis and ovary. The functions of most of these genes are undetermined and thus they represent a candidate pool of genes that could play important roles in reproduction-related processes. Those that have been discovered, such as syncytin, PEG10, and RTL1 have profoundly changed our understanding of mammalian reproduction. However, these discoveries were largely coincidence. Part of the significance of this application is our evolutionary-guided strategy that intentionally focuses on discovering gag-like capsid genes that could have been domesticated for roles in fertility. Our data provides strong evidence that the approach is working. These studies will not only add PNMA genes to the small but growing list of retrotransposon-derived genes that are critical for fertility but also provide mechanistic insight for how they participate in this process. Effects of ectopically expressed capsid proteins in tumors have been characterized, but very little is known regarding their physiological functions. Beyond their possible roles in germ cell development, the associations of these proteins with pro-apoptotic and pro-survival factors suggest that they could play a role in oocyte attrition which adversely affects many females wanting to have children.

Until recently, functional understanding of capsid genes has been held back because the sequences that encode them were dismissed as ‘junk’ repetitive DNA. An enormous phylogenetic study by the Wiedenheft lab revealed seventeen full-length Metaviridae capsid genes conserved across ~150 million years of mammalian evolution (detailed below). These analyses have served as a springboard for the hypothesis-driven genetic and molecular studies proposed here to determine the germ cell functions of these genes. Our data support the idea that at least two capsid genes are crucial for mammalian fertility. Furthermore, we have determined that the proteins encoded by these genes have retained the ability to form capsids and a structural evaluation of these capsids is underway. Together, these studies will provide a profound resolution of reproductive functions governed by ancient capsid genes from the evolutionary to the atomic level.

### Domesticated capsids as pro-fertility factors

As implied by their name, gag capsid genes encode proteins that form large hollow structures within which biomolecular cargo, such as RNA, is housed and transported. The roles of some Metaviridae-derived gag proteins are known, and the ability to form a capsid is a crucial part of their function. One example is ARC, which serves as a delivery vehicle to shuttle mRNA across neuronal synapses to regulate learning and memory. Our data demonstrate that two capsid genes, which are robustly expressed in reproductive tissues where they appear to play important roles in fertility maintenance, have retained the capacity to form capsids that exit cells. This has led to the provocative hypothesis that these capsids are functional — possibly by promoting intracellular communication within the testis and/or ovary. The idea that capsid proteins encoded in the human genome could be repurposed as a delivery vector for mRNA (such as vaccines) is well-founded and is being pursued by other bioengineer-focused groups. Our data provide a basis for the exciting speculation that capsids studied here could be isolated and their cargo (either endogenous or designer) be delivered into testes and/or ovaries as a pro-fertility treatment.

### Identify the fertility-associated processes mediated by PNMA1 and PNMA4

Retrotransposons and genes derived from them are often highly expressed in reproductive tissues, particularly during germ cell development. A critical challenge is to understand whether and how these genes are influencing human reproductive biology. Based on our data, we hypothesize that two Metaviridae-derived genes — PNMA1 and PNMA4 — are serving key functions in testes and ovaries. In this aim we will determine the fertility-related processes that are affected by these genes.

### Conservation of Metaviridae-derived genes provides a basis for function

Metaviridae are a subclass of long terminal repeat (LTR) retrotransposons. The life cycle of an LTR retrotransposon is much like a retrovirus except it occurs entirely within the cell. It is transcribed into an mRNA which encodes two polyproteins: gag and gag-pol. Gag is the precursor to the capsid and nucleocapsid proteins and pol is precursor to a protease, a reverse transcriptase-RNaseH, and an integrase. Gag and gag-pol proteins assemble with retrotransposon mRNA into a capsid where polyproteins are processed, and mRNA is reverse transcribed into cDNA. This cDNA copy is bound to integrase proteins and can re-enter the nucleus to integrate in the genome at another locus.

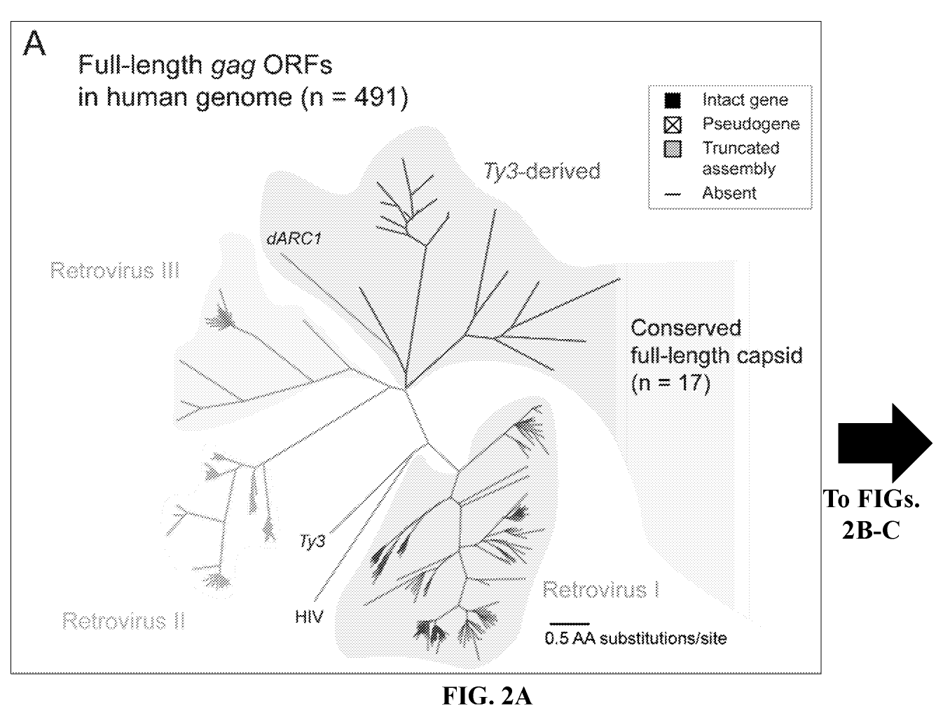

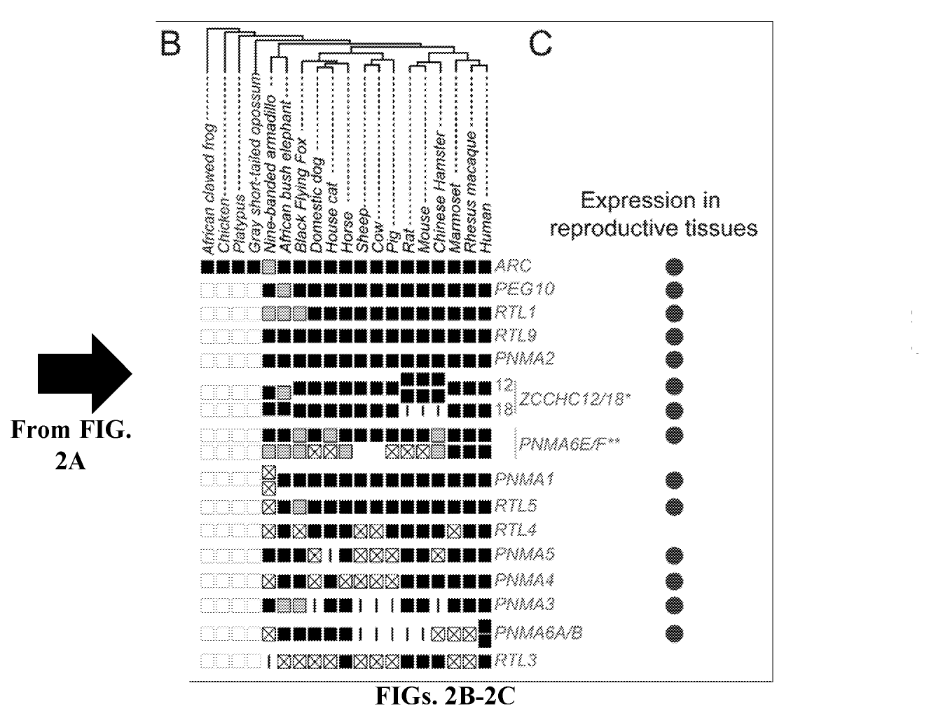

To assess the extent of capsid gene domestication in humans, we identified all gag-like genes encoded in the human genome. Guided by atomic structures and AlphaFold predictions, we built Hidden Markov Models (HMMs) for gag-like capsid genes from retroviruses/endogenous retroviruses (ERVs) and the three major clades of LTR retrotransposons: Metaviridae (Ty3), Pseudoviridae (Ty1/copia), and Belpaoviridae. Using these HMMs, we queried the telomere-to-telomere human genome and we identified 5,786 gag-like genes from retroviruses and Metaviridae, but none from Pseudoviridae or Belpaoviridae. 491 of these gag genes encode a full-length capsid domain, and we constructed a maximum-likelihood phylogenetic tree designating their evolutionary relationships (FIG. 2A). Branch length analysis indicated that Metaviridae capsid genes within the human genome are far more divergent than retroviral capsid genes. This divergence is likely indicative of ancient gene retention and purifying selection, both of which support the hypothesis that these genes have been conserved based on a function.

To identify which human Metaviridae-derived capsid genes are conserved among mammals, we searched for orthologous genes in 16 species representative of mammalian diversity (14 placental mammals, one marsupial, and one monotreme, one bird, and one amphibian). We identified nine full-length Metaviridae capsid genes present in the human genome that are universally retained in placentals. Eight others have been intermittently lost or pseudogenized (FIG. 2B). We next analyzed the expression patterns of these genes in humans (gtexproject.org). Strikingly, most conserved capsid genes are robustly expressed in male and female reproductive tissues (FIG. 2C). These data support the hypothesis that several Metaviridae-derived gag-like capsid genes have evolved to function in reproductive tissues as fertility factors.

### Expression of gag-like genes in reproductive tissues is governed by master meiotic regulators

If gag-like capsid genes have been retained in mammals for reproductive functions- a clear prediction is that animals lacking one or more of these genes will have fertility-related phenotypes. For cost and labor reasons, we could not generate and characterize seventeen mouse knockouts (KOs). Instead, we needed to identify ideal candidates which began by assessing which gag genes exhibited robust and regulated expression in testis and/or ovary. By analyzing published RNAseq data from mouse testis, we found two — Pnma1 and Pnma4 — which showed the highest upregulation (compared to other candidates) in spermatocytes.

A brief introduction to PNMA genes: The paraneoplastic antigen Ma (PNMA) gene familyincludes six primary members in humans PNMA1-3 were identified as autoantigens present in the brains of paraneoplastic disorder (PND) patients. PNMA4-6 were identified as having high sequence similarity to PNMA1-3 but are not implicated in PND. Expression of PNMA-family proteins tends to be restricted to sites of immune privilege, such as the testis and ovary, possibly due to their autoantigenic properties. Knockdown of Pnma5 in mouse oocytes results in severe meiotic progression defects through an unknown mechanism.

From published mouse ChIP-seq data, we found evidence that both Pnma1 and Pnma4 are regulated by master germ cell transcription factors: STRA8 (required for meiotic initiation) binds upstream of Pnma4 and MYBL1 (required for progression through pachynema) binds upstream of Pnma1. Published mRNA expression data from Stra8 -/- spermatocytes and Mybl1 -/- testes indicated that Pnma4 transcript abundance is significantly reduced in the absence of Stra8. Both Pnma1 and Pnma4 are reduced in the absence of Mybl1. These data support the idea that transcription of Pnma1 and Pnma4 is developmentally regulated during meiosis. In placental mammals, there has been substantial expansion of the PNMA gene family. We tested whether meiotic transcription of PNMA can be observed in marsupials which did not experience this expansion and harbor only a single PNMA gene: M-PNMA. From published opossum RNA sequencing data, we found that M-PNMA is highly and specifically upregulated in spermatocytes during pachynema. These data support the hypothesis that meiotic expression of PNMA was either present in the common therian ancestor or has evolved independently in both placentals and marsupials.

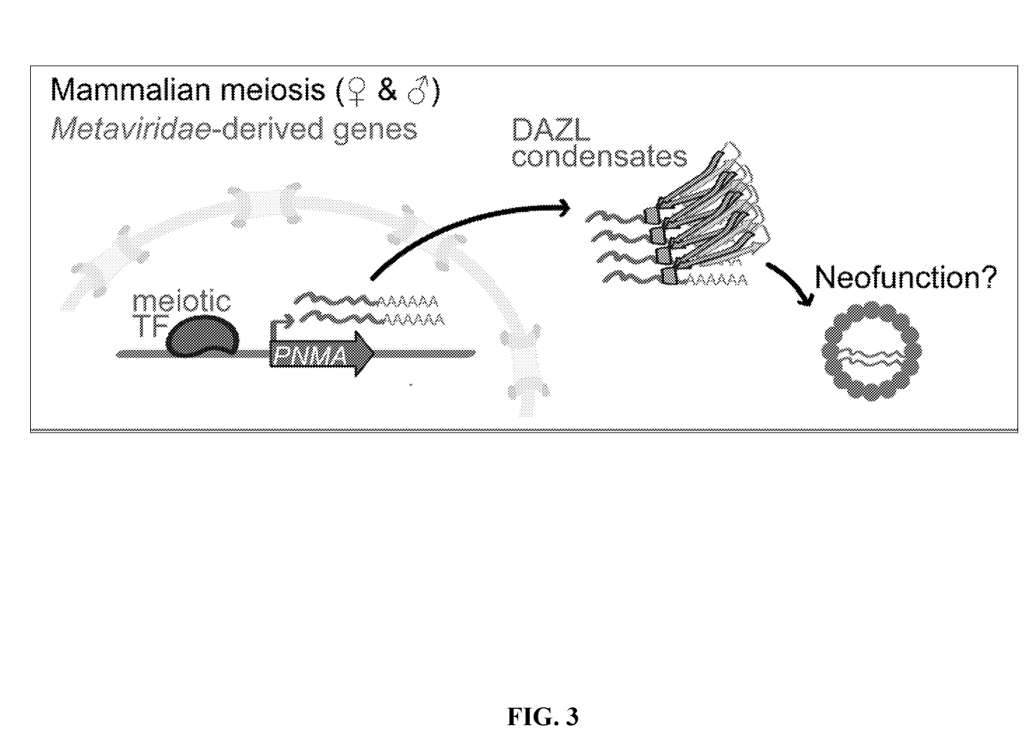

Yeast cells use Rim4 condensates to block translation of Metaviridae elements that become expressed during meiosis. The mammalian analogs of Rim4 are the DAZ (Deleted in Azoospermia) and DAZL (DAZ-like) proteins: all are essential for germ cell development, affect gene expression post-transcriptionally by regulating translation, and form higher-order condensates in vivo. In human meiotic ovaries and mouse spermatocytes, DAZL binds specifically to the 3´UTRs of PNMA1 and PNMA4. We found that DAZL binding enhances the translation of PNMA1 and represses that of PNMA4, and mutating DAZ binding sites in PNMA 3´UTRs prevents DAZ regulation. Thus, germ cell expression of PNMA1 and PNMA4 is developmentally regulated at multiple levels (FIG. 3) — but are these genes serving important reproductive functions?

### Mice lacking Pnma1 or Pnma4 are subfertile

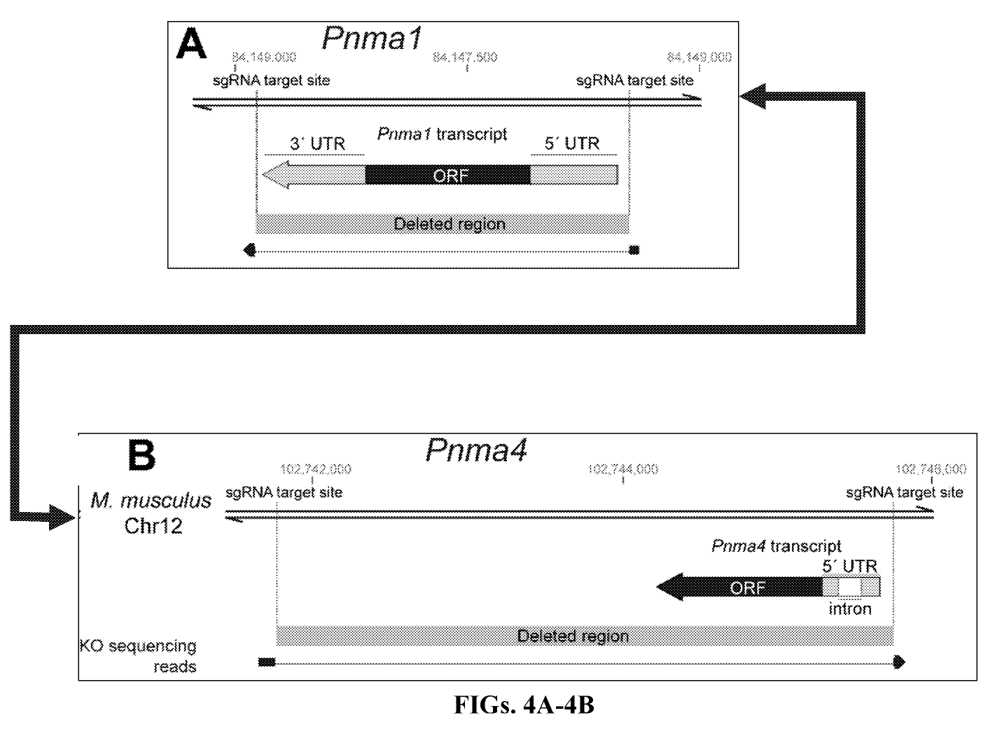

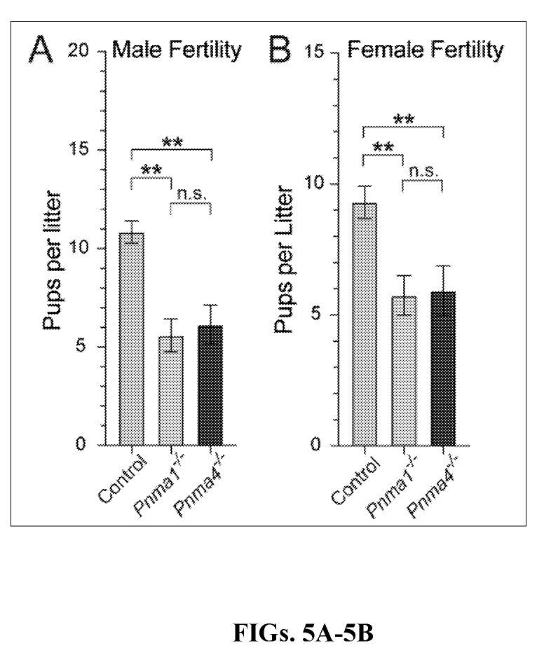

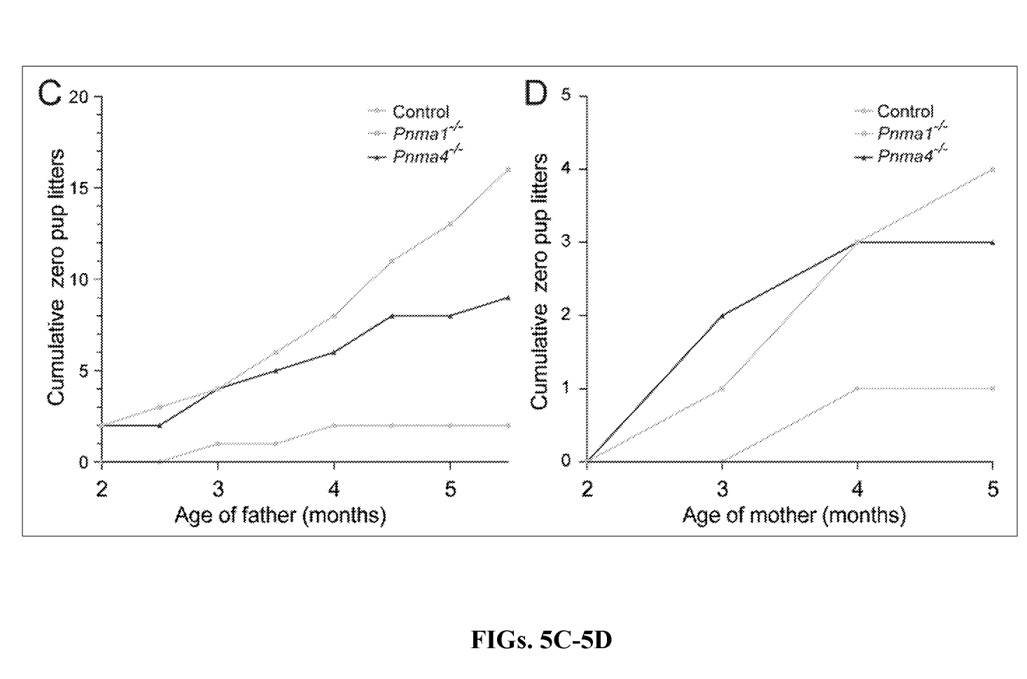

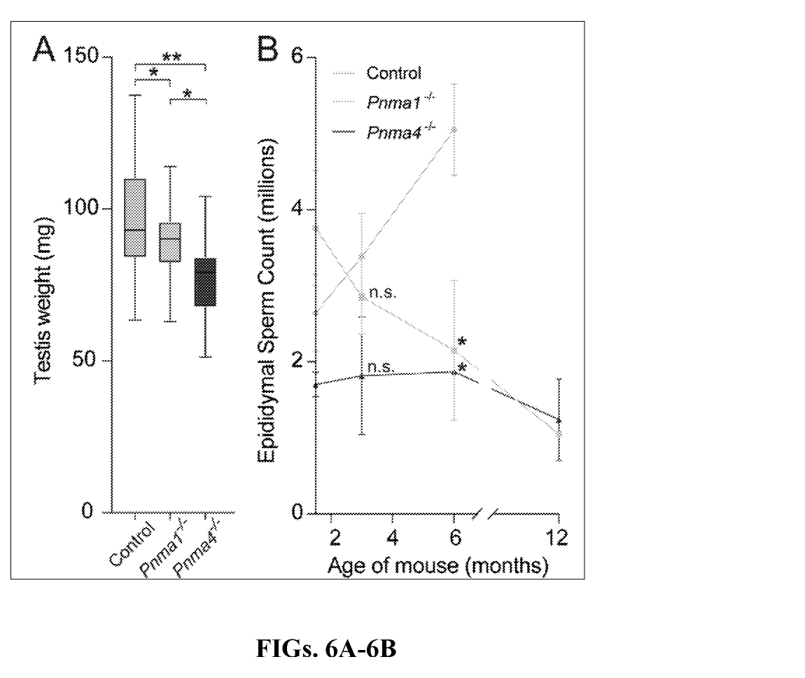

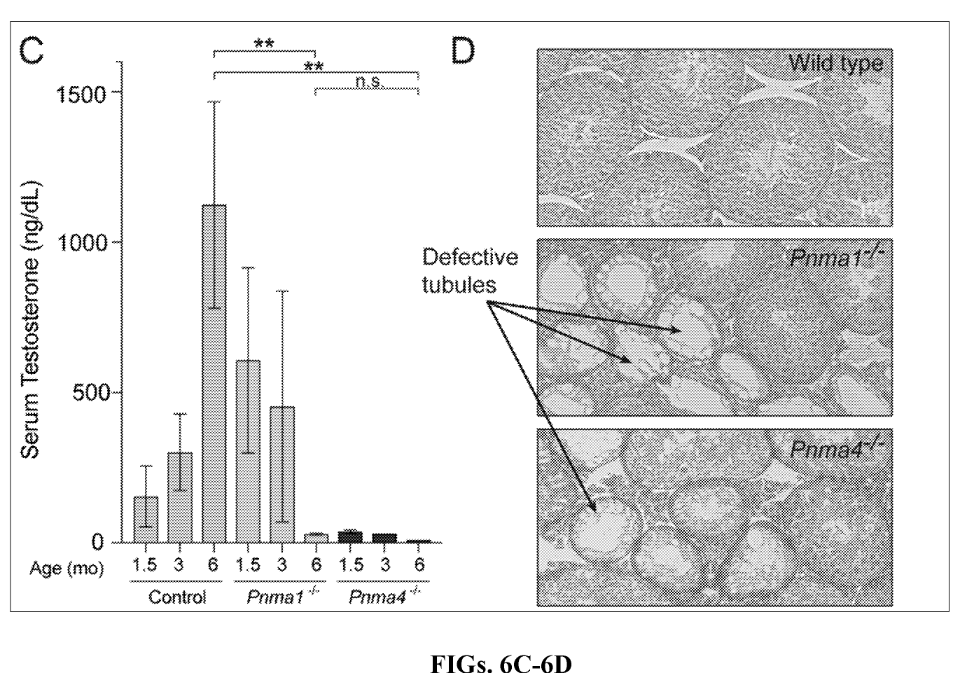

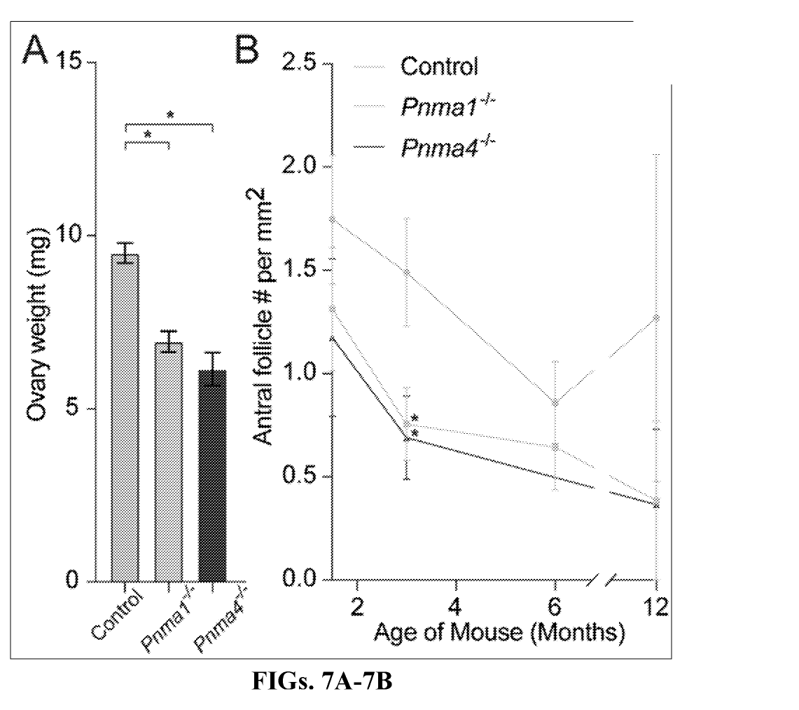

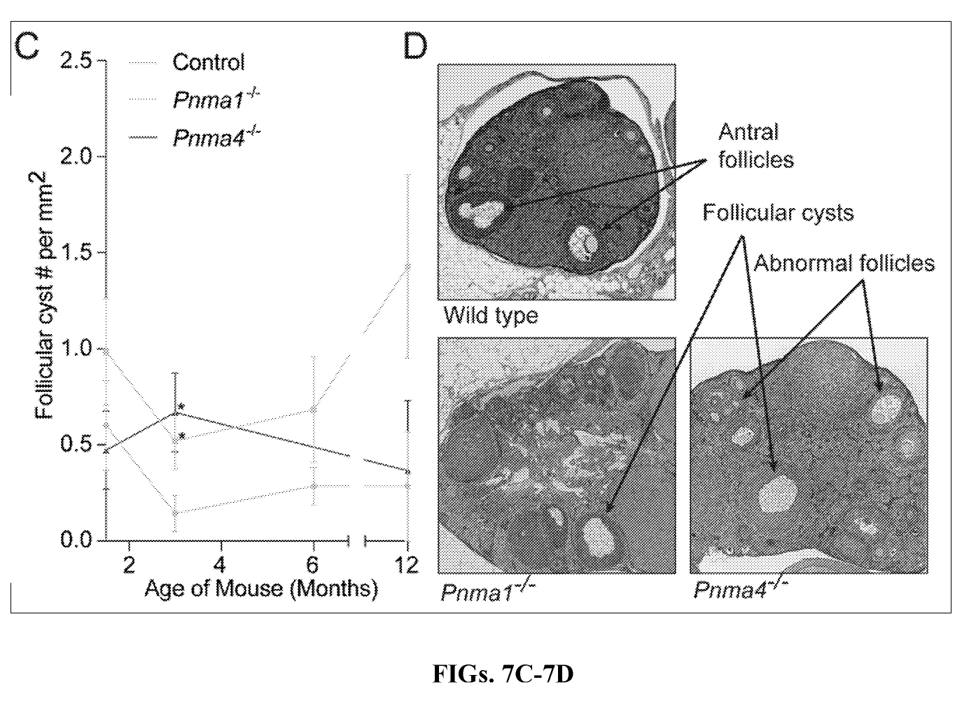

To test the hypothesis that germ cell expression of Pnma1 and/or Pnma4 is providing a reproductive function, we generated clean mouse KOs (null alleles) using CRISPR-Cas9, which we verified by sequencing (FIGS. 4A, B). We were initially concerned that Pnma4 KO mice were reported to be fertile without significant developmental defects. Furthermore, bulk phenotyping a Pnma1 KO mouse (available on the International Mouse Phenotyping Consortium) reported no major fertility defects. While these results likely rule out total infertility, subtler defects or subfertility could easily have gone unnoticed. An assessment of fertility showed that Pnma1 -/- and Pnma4 -/- are subfertile as assessed by pups per litter (FIGS. 5A, B). Notably, the number of zero pup litters in mutant crosses steadily increased with age from (1.5 to 6 months) compared to control crosses (FIGS. 5C, D). We conclude that while PNMA1 and PNMA4 are not required for male or female fertility, they are important for normal fertility, and they could contribute to the maintenance of fertility with age. An assessment of reproductive phenotypes in male animals revealed that testis weight was lower in mutants compared to controls (FIG. 6A). Correspondingly, we found that mutant sperm counts declined with age (FIG. 6B). Furthermore, serum testosterone levels (measured by the ligand core laboratory at University of Virginia) in Pnma1 -/- were similar to control at 1.5 and 3 months, but dramatically declined 6 months (FIG. 6C). Pnma4 -/- mice showed over 10-fold lower serum testosterone levels compared to controls at all time points. A histological analysis of sectioned testes revealed that many of the seminiferous tubules in Pnma1 -/- and Pnma4 -/- were devoid of spermatocytes, spermatogonia, and spermatids (FIG. 6C). Peritubular myoid and Sertoli cells appeared normal. The empty tubules explain the decreased testis weight and sperm counts while the presence of some normal tubules may clarify why these mice are not infertile. These phenotypes are hallmarks of spermatogenesis mutants and likely underlie the mutants’ subfertility. Our assessment of reproductive phenotypes in Pnma1 -/- and Pnma4 -/-females revealed abnormal ovarian morphology. While we are still in the early stages of quantifying and interpreting these data, we observed that mutantsexhibit decreased mean ovary weight and antral follicle count (FIGS. 7A, B) with a corresponding increase in follicular cyst formation (FIGS. 7C, D). These cysts often showed over-proliferation of granulosa cells and are reminiscent of cysts in female mice lacking estrogen or FSH hormone receptors. Antral follicle decline appears to worsen with age which may underlie the age-based increase in zero pup litters we observe in these mutants.

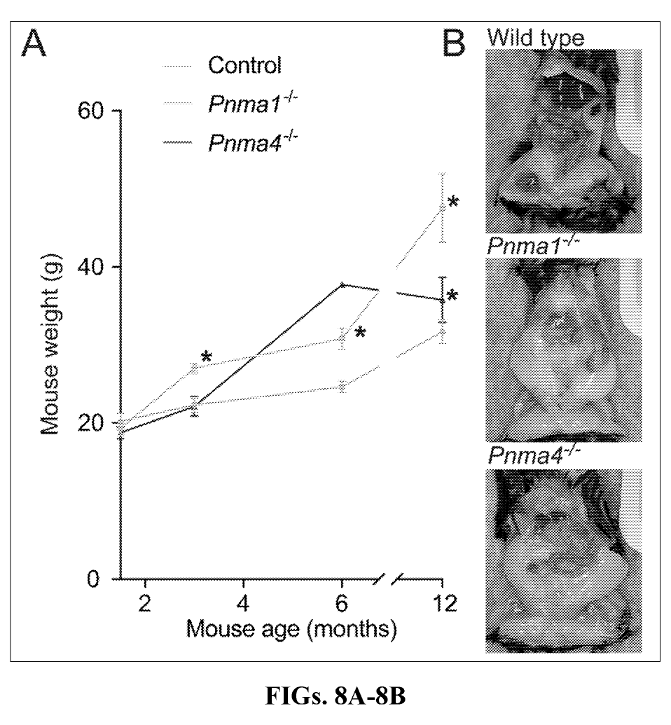

At ~4 months, it became evident that mutant animals, housed and fed under identical conditions to wild type littermates, were obese. Body weights of Pnma1 -/- and Pnma4 -/- were significantly higher than controls with disproportionate abdominal fat accumulation (FIGS. 8A, B). This obesity phenotype appears to be age-dependent and may be related to accelerated loss of reproductive capacity. A similar phenotype is observed post-ovariectomy or in mice deleted for female reproductive hormone receptors. In summary, Pnma1 -/- and Pnma4 -/- animals have indications of defective male and female reproductive function and investigation into the mechanistic bases underlying these phenotypes will yield important insights.

### Delineate the roles of Pnma1 and Pnma4 in male fertility and germ cell development

All experimental genotypes will be compared to wild type and heterozygous (+/-) controls, and all genotypes will be analyzed in triplicate (at minimum) to ensure rigor and reproducibility.

General subfertility assessment: To understand the extent to which PNMA1 and PNMA4 contribute to fertility and its maintenance with age, we will continue our aging time course analysis of male fertility in single and double KO mice. In these trials, six breeding pairs (for each mutant) between mutant males and wild-type females will be monitored for 7 breeding rounds over a 6-month period. We will determine % pregnancy, duration between litters, and litter size from plugged females. We will also continue our analysis of testis weight, serum testosterone, and sperm count at 1.5, 3, 6, and 9 months (from cauda epididymis) of mutants which, when compared to controls, can indicate spermatogenesis defects. Lastly, we will determine whether transmission of mutant alleles is Mendelian.

Histological analysis of testes: Sexual reproduction relies on the production of gametes (i.e., sperm and eggs) that contain half the DNA complement of their parental progenitors. To understand the roles of Pnma1 and Pnma4 in male gametogenesis, we will conduct seminiferous tubule staging of single mutant, double mutant, and wild type males in fixed adult (1.5, 3, 6, and 9 month) testes sections stained with Periodic acid Schiff (PAS) and hematoxylin. We will quantify the proportion of spermatogenic and post-meiotic cell types to determine which specific step is disrupted⁵¹. We will also analyze Leydig cell count/morphology. As mentioned above, analysis of Pnma1 -/- and Pnma4 -/- testes tissue sections revealed a phenotype in which up to half of the tubules were devoid with neighboring tubules appearing wild type. This unusual phenotype could be explained by age-dependent spermatogonial stem cell (SSC) loss. To test this idea, we will stain tissue sections using SSC markers such as CD2 and PIWIL4. Lastly, we will perform TUNEL staining in sections from single/double mutants (and controls) to determine whether traditional apoptotic cell death pathways are implicated in the abnormal testicular morphology observed in these animals.

Analysis of male meiosis: Because male expression of both Pnma1 and Pnma4 peaks in pachynema²⁸, an in-depth cell biological analysis of meiosis in Pnma1 -/- and Pnma4 -/- single and double mutants will be informative. Decades of research has yielded cytological and histological markers that allow monitoring of meiotic events. Deviation from wild type staining patterns often reveals specific insights regarding function which can be further tested. We will assess meiotic chromosome segregation by DAPI-staining meiotic nuclei and assessing anaphase bridging and lagging chromosomes. If these analyses indicate meiotic defects, we will further analyze chromosome segregation using tubulin and centromere immunofluorescence (IF) on testis sections. To specifically assess meiotic prophase I defects, we will immunostain chromosome spreads prepared from spermatocyte nuclei for the chromosome axis protein SYCP3 (Synaptonemal Complex Protein 3) from adult male mice. SYCP3-positive axes begin to appear in leptonema, align with their homolog in zygonema, are completely paired in pachynema, and separate in diplonema. Deviations from wild type SYCP3 staining patterns are indicative of defects in processes such as chromosome condensation, pairing, and recombination. To assess meiotic recombination, we will analyze the sequential appearance of recombination proteins on chromosomes including foci containing γ-H2AX, DMC1, RAD51, MSH4/5, and MLH1/3 which mark the sites of programmed double strand breaks and recombination intermediates. We will also measure localization of proteins such as BRCA1, and ATR to XY chromosomes which silence transcription from these chromosomes and may prevent production of toxic sex-chromosome products. These experiments will provide a broad overview of the male meiotic processes involving Pnma1 and Pnma4.

### Delineate the roles of Pnma1 and Pnma4 in oogenesis and female fertility

Assessment of subfertility and ovarian morphology: Pnma1 and Pnma4 are expressed in ovary and our data support the notion that both genes are likely important for female fertility. To begin to understand the roles of Pnma1 and Pnma4 in females, we will measure fertility, oogenesis/meiosis, and oocyte attrition in Pnma1 -/- and Pnma4 -/- single mutants, double mutant, and control mice. We will assess fertility in females by the fertility breeding schemes outlined for males in aim 1a. We will also examine length of estrous cycles, serum steroid hormone levels (estradiol, progesterone, and testosterone), and peptide hormone levels (FSH, LSH, and anti-Mullerian hormone (AMH)) in mutant and wild type animals at 1.5, 3, 6, and 9 months.

We will continue our analysis of ovarian morphology in fixed, sectioned, and stained tissues obtained from mice aged 1.5, 3, and 6, and 9 months. We will measure primordial, primary, secondary, and antral follicles, follicular atresia, corpus lutea, and cyst formation (per unit area). To assess ovarian reserve, we will measure oocytes by immunostaining sectioned ovaries with the germ cell marker MSY2 which is an oocyte-specific RNA-binding protein that serves as a scorable oocyte marker. The ovarian and obesity phenotypes observed in Pnma1 -/- and Pnma4 -/- mutants are reminiscent of a defect in either production of, or response to, estradiol. To test this idea, we will determine if mutant phenotypes, such as obesity and fertility, can be rescued by estradiol replacement therapy administered by subcutaneous injection.

Female germ cell analysis: While the ovarian morphology defects that we observe in Pnma1 -/- and Pnma4 -/- mutants do not necessarily point to germ cell failure, the fact that Pnma5 knockdown results in meiotic failure¹⁷ and that male PNMA expression peaks in pachynema suggest to us that it will be worthwhile to analyze female meiotic progression. The study of female germ cells is more challenging than in males due in large part to the fact that female meiosis is initiated during embryogenesis (E13.5) and pachynema is complete around birth. To achieve this, we will isolate fetal ovaries and analyze chromosome segregation in DAPI-stained meiotic nuclei. We will also immunostain chromosome spreads for meiotic markers such as DMC1, SYCP3, and MLH1/3 as detailed in aim 2a. For each experiment we will sacrifice pregnant mothers and each ~8 pup litter from a double heterozygous cross will yield, on average, one mutant female and two heterozygous controls. As a parallel approach, we will assess meiotic chromosome segregation in live-imaged oocytes collected from mutant and control animals. Oocytes collected in this manner readily undergo meiosis and we will analyze these cells with collaborative support (see Karen Schindler and Binyam Mogessie letters of support). These experiments will provide a comprehensive overview of the female meiotic processes involving Pnma1 and Pnma4.

### Dissect the molecular mechanisms underlying PNMA1 and PNMA4 function

Rationale: PNMA1 and PNMA4 evolved from gag sequences⁴. Retrotransposon gag proteins have two primary functions: bind RNA cargos and package these payloads in capsid structures for inter/intra-cellular delivery⁴. Domesticated proteins derived from gag often bind to nucleic acids such as the ARC protein which shuttles mRNA across synapses⁶⁶. We hypothesize that PNMA1 and PNMA4 will utilize nucleic acid binding and/or accessory protein co-factors such as pro-apoptotic factors to carry out their functions. The following aim is designed to test these ideas and to assess key properties of PNMA proteins such as their spatiotemporal localization. Successful completion of these experiments, when combined with the fertility and reproductive physiology analyses performed in Aim 1, will reveal the molecular mechanisms underlying PNMA function.

### Determine the subcellular and temporal expression patterns of PNMA1 and PNMA4

Localization analysis: Understanding the localization and expression patterns of PNMA1 and PNMA4 which will inform function is an initial step. Fortunately, monoclonal IF-grade antibodies are commercially available for both proteins. We will analyze their tissue and cell-specific localization patterns using immunostaining analysis of sectioned testes from (~3 and ~6-month adults) and ovaries (E16.5 and adult) with α-PNMA1 and α-PNMA4 antibodies. For immunoblot and IF assays, we will utilize knockout mice as controls to make sure that our signal is derived from Pnma1 and/or Pnma4. As a secondary approach, we will use subcellular fractionation of testis and ovary lysates followed by immunoblot to determine whether PNMA1 and PNMA4 are expressed in the cytoplasm, nuclear, mitochondrial, extracellular matrix, and/or specific membrane fractions. Lastly, because both Pnma1 and Pnma4 are transcriptionally upregulated during pachynema, it is possible that they are playing direct roles in meiotic recombination. If we find that that PNMA1 and/or PNMA4 are nuclear, we will analyze whether and how they localize on male and female meiotic chromosome spreads by immunofluorescence. These experiments will allow us to determine (a) which cell types express PNMA1 and PNMA4, (b) their temporal expression patterns, and (c) where in the cell they are being expressed.

### Determine the binding partners and nucleic acid targets of PNMA1 and PNMA4

Protein co-factor analysis: PNMA1 and PNMA4 have been implicated in apoptosis pathways in the context of ectopic expression in cancer cells. We will test whether these functions are relevant to reproductive tissues where PNMA1 and PNMA4 are physiologically expressed. PNMA4 protein interacts with the pro-apoptotic protein Bax and the pro-survival proteins Bcl-2 and Bcl-X(L) and can mediate caspase-dependent apoptosis. A pro-apoptotic function could be important for oocyte development during which more than two thirds of meiotic prophase oocytes are eliminated before birth and could possibly represent a quality control mechanism that exists to eliminate unfit oocytes. On the other hand, a pro-survival role could explain the partial penetrance phenotype we observe in mutant testicular tubules. If PNMA1 and/or PNMA4 proteins function as apoptotic or survival factors in reproductive tissues, a clear prediction is that they will also interact with these factors in developing germ cells. To test this prediction and to determine other important binding partners, we will immunopurify (IP) PNMA1 and PNMA4 from 30-week adult testes, fetal E16.5 ovaries, and adult ovaries at ~6 months. We will then identify bound co-factors by mass spectrometry and validate all key factors by reciprocal Co-IP (see Marko Jovanovic letter of support). If PNMA1 and/or PNMA4 are found to be bound to apoptosis factors in developing gametes we will prioritize further study of this function.

Nucleic Acid binding analysis: The gag-like nature along with the presence of an N-terminal RRM on both PNMA1 and PNMA4 has led us to hypothesize that these proteins bind nucleic acids. To test this hypothesis in vivo, we will IP both proteins from UV-crosslinked adult testes (~6 month) and E16.5 ovaries, determine DNA binding using a DNA-specific dye such as YOYO-1 and RNA binding using an RNA-specific dye such as SYTO RNAselect. In parallel, we will conduct polynucleotide kinase labeling assays in which we label free ends of bound nucleic acids with radiolabeled ATP. We will combine these assays with control DNase and/or RNase treatments to determine whether PNMA1 and PNMA4 bind to DNA and/or RNA. If our assays reveal DNA binding for PNMA1 and/or PNMA4, we will determine DNA binding sites by sequencing immunoprecipitated chromatin (ChIP-seq). ChIP-seq will be abandoned (for a protein) if we do not observe nuclear staining. If we observe RNA-binding for one or both proteins, we will perform high-throughput sequencing of RNA isolated by crosslinking immunoprecipitation (HITS-CLIP, also known as CLIP-seq) to determine PNMA1 and/or PNMA4 RNA binding targets. Importantly, we can conduct control ChIP and/or HITS-CLIP analyses using Pnma1 -/- and/or Pnma4 -/- KO mice which will be useful to determine bona fide signal in these assays. These approaches will reveal candidate genes that are likely regulated by PNMA1 and PNMA4. In validation and follow-up studies, we will co-express PNMA1 and PNMA4 along with their putative targets in cultured oocytes (collaboration with Binyam Mogessie, see letter of support) which will allow us to mutate target binding sites and evaluate the effects of these mutations on gene expression. From these experiments, we will gain a comprehensive understanding of the protein and nucleic acid binding partners of PNMA1 and PNMA4.

### Determine the structure and function of PNMA capsids

Rationale: Gag family proteins self-assemble into capsid-like structures that are critical for selective packaging and inter/intra cellular delivery of molecular cargo. For example, ARC and PEG10 are domesticated Metaviridae gag-like proteins that have preserved capsid assembly because it is critical for their function. Like ARC and PEG10, our data (and published reports) suggest that PNMA proteins have retained their intrinsic property to self-assemble into capsids. We hypothesize that capsid formation is important for PNMA function. To test this hypothesis, we will a) isolate PNMA capsids from mouse testes and ovaries, and human cells expressing PNMA1 or PNMA4, b) identify their cargo using a combination of mass spectrometry and sequencing, and c) determine their structures by single particle cryo-EM. This information will inform experiments (discussed below) designed to assess capsid function.

### PNMA proteins form capsid-like structures that exit human cells

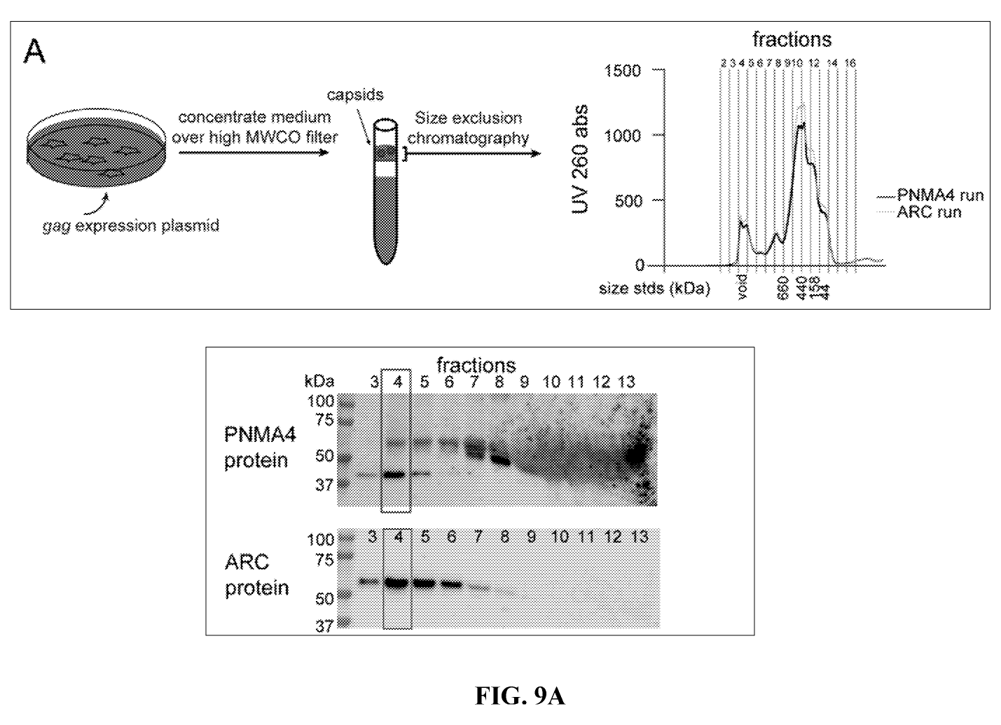

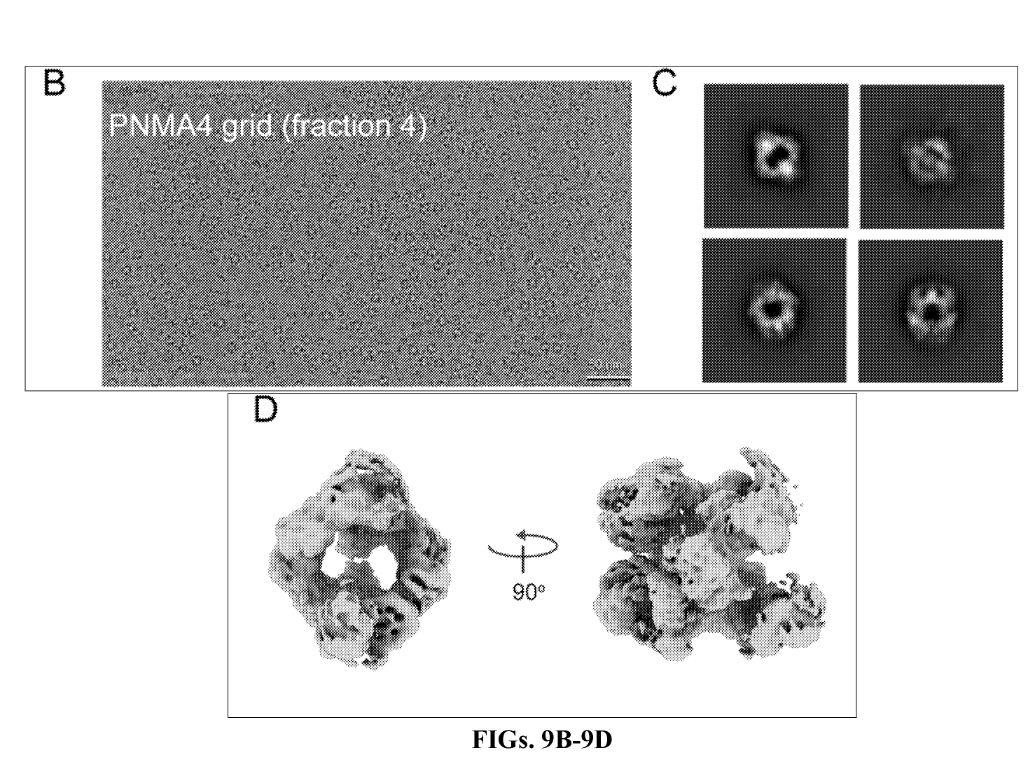

Metaviridae capsids are massive (Ty3 capsids contain 540 gag protein subunits and are ~ 20 megadaltons), which makes them amenable to isolation by size-based fractionation. To validate our approach and to verify that PNMA1 and/or PNMA4 have the capacity to form capsids, we expressed V5-tagged PNMA1/4 (with a positive control ARC) in human HEK-293T cells and fractionated cell lysate over a density gradient. We found that both PNMA1 and PNMA4 form high-molecular weight assemblies that are consistent with capsid formation. To determine if these capsid-like structures have the capacity to exit cells, we again expressed V5-tagged PNMA4 and ARC in HEK-293T cells. Instead of collecting cells, we instead filtered the growth medium and removed the cells. We concentrated the medium over a 100 kDa molecular weight cutoff (MWCO) filter, fractionated the concentrate by size-exclusion chromatography, and analyzed the fractions by immunoblot (FIG. 9A). We observed enrichment of both PNMA4 and ARC in a high molecular weight fraction, and we visualized this fraction by transmission electron microscopy (TEM). Fractions corresponding to the assembled complex were spotted on mesh, plunge frozen, and screened at MSU’s cryo-EM core facility. We observed capsid-like structures generated in both the PNMA4 and ARC samples (FIG. 9B). SerialEM software was used for automated data collection. Frames of each movie were aligned, and Contrast transfer function (CTF) parameters estimated in cryoSPARC. Subsequently, ~2.8 million particles were picked using CryoSPARC’s template picker, which were used for reference-free two-dimensional (2-D) classification into 50 classes (FIG. 9C). Only a single volume displayed distinct structural features in heterogeneous refinement, and particles corresponding to this volume were subsequently refined (n = 765,573 particles) to a reported ~4.5 Å resolution (at the gold standard FSC threshold 0.143) (FIG. 9D). This preliminary reconstruction reveals spherical architecture consistent with capsid formation, but the resolution is currently insufficient to accurately model the individual PNMA subunits. Notably the result that PNMA proteins have the capacity to form capsid-like structures that exit cells has been corroborated by a published report. Together, these results support the idea that PNMA proteins have retained the capability to form capsids that exit cells.

### Isolate PNMA capsids from testis and/or ovary

To assess the in vivo capsid-forming potential of PNMA1 and PNMA4, we will enrich capsids from mouse testis and ovary tissue lysate by ultracentrifugation on a sucrose cushion. We will then re-fractionate the enriched pellets on 10-40% sucrose density gradients. As a parallel approach, we will purify PNMA1 and PNMA4 from wild type testis and ovary lysate (or from pre-fractionated samples) by IP using commercial antibodies. We will test for the presence of PNMA protein using immunoblotting of fractions and, subsequently, we will conduct TEM on high molecular weight fractions containing PNMA protein. Lysates from null mice will serve as critical negative controls that will give us confidence that what we observe depends on functional a Pnma1 or Pnma4 gene. If we observe capsids, we will go on to determine their nucleic acid cargo by sequencing and protein cargo by mass spectrometry and solve their structure by cryo-EM.

### Structure-function analysis of PNMA capsids

Structural analyses: Our goals are to understand the role of PNMA capsids in fertility and, in time, to use these insights to develop capsids for therapeutic applications. We aim to determine high-resolution structures of PNMA1 and PNMA4 capsids using single-particle cryo-EM. Structure is intrinsically linked to function, so a high-resolution structure of these capsids will be critical to understanding how capsid proteins self-assemble into higher order oligomeric structures that selectively package specific payloads. Ongoing efforts are aimed at improving the resolution of the preliminary PNMA4 capsid subunits, using two parallel strategies. First, we are using our preliminary reconstruction as a template for iterative rounds of particle picking, which will aid successive rounds of 2D and 3D classification, followed by multi-class ab initio reconstruction and heterogenous refinement.

In parallel, we will continue to improve the sample preparation. While the initial reconstruction is encouraging, the sample is heterogeneous, which has complicated structure determination and limited the resolution. Ongoing efforts are designed to incorporate antibody pulldowns into our purification. Commercially available monoclonal antibodies to PNMA1 and PNMA4 will be used for IP, followed by size exclusion chromatography using a Sephacryl S-500 column which is designed for preparative size exclusion chromatography of large macromolecules. We anticipate that these ongoing improvements will result in high-resolution structures of PNMA1 and PNMA4 subunits and capsids, and that these structures will clarify the assembly principles of these capsids.

### EXPERIMENTAL - II

### Expression of PNMA1 and PNMA4 declines with age in human ovaries

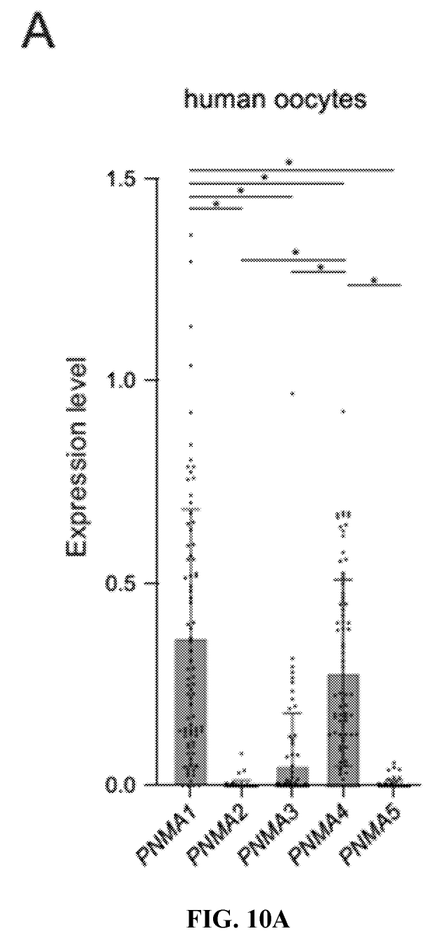

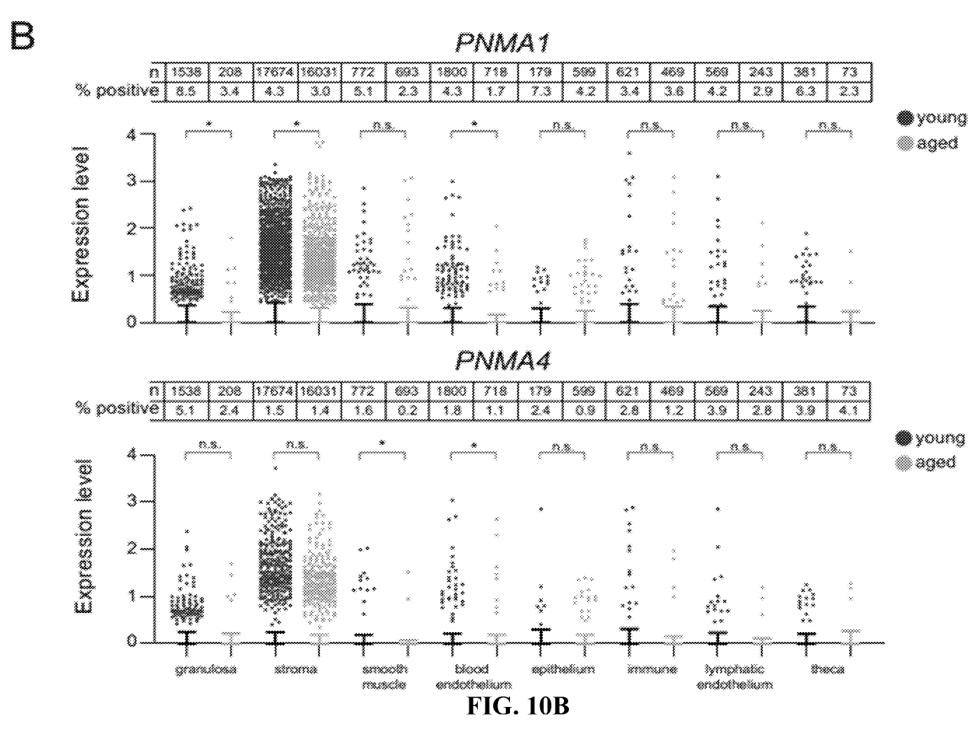

While previous studies of mouse male gonads indicated that both PNMA1 and PNMA4 are expressed at their highest levels in spermatocytes³⁷, we sought to determine which and to what degree human female gonadal cells express PNMA-family genes. We analyzed PNMA1-5 expression by single-nuclei RNAseq in human ovaries donated from four young individuals in their 20s, four older individuals in their late 40s to early 50s, and in published human oocyte expression data^{38,39}. Among PNMA1-5, PNMA1 and PNMA4 showed the highest expression levels throughout the ovary (FIGS. 10A, 10B). Oocytes expressed the highest levels of PNMA1 and PNMA4. The somatic ovarian cell type that expressed the highest levels of either gene was granulosa cells: in young adult ovaries, 8.5% of granulosa cells expressed PNMA1 and 5.1% expressed PNMA4, possibly indicating specific granulosa subpopulations (FIG. 10B). Comparing younger and older ovaries, PNMA1 expression was significantly higher in young granulosa, stroma, and blood endothelium cells, while PNMA4 was significantly higher in young smooth muscle cells signifying age-related decline in expression of these genes.

In mouse spermatocytes and human fetal ovaries, the 3’ untranslated regions (UTRs) of PNMA1 and PNMA4 transcripts are bound by DAZL^{40,41}, which can activate or repress mRNA translation⁴². To test whether the PNMA-DAZL interaction could be of regulatory significance, we expressed human PNMA1, PNMA4, or SMC1b (control DAZL target) with or without DAZL in human cells (HEK293T). DAZL augmented PNMA1 protein levels and repressed PNMA4 without altering mRNA levels supporting the idea that DAZL can regulate the translation of PNMA transcripts.

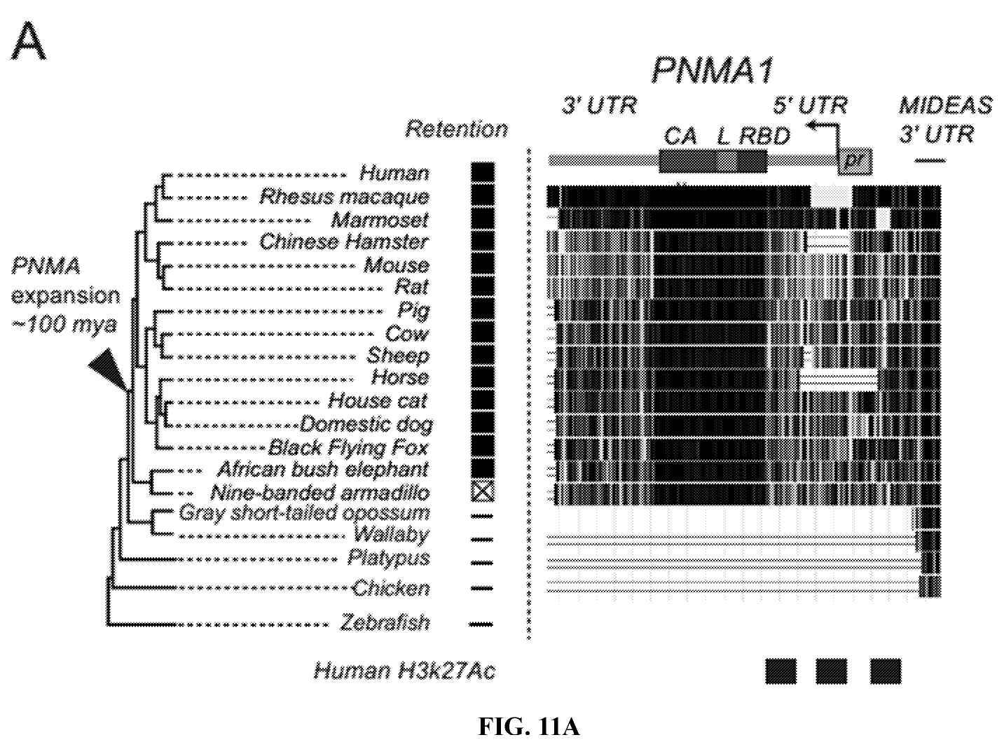

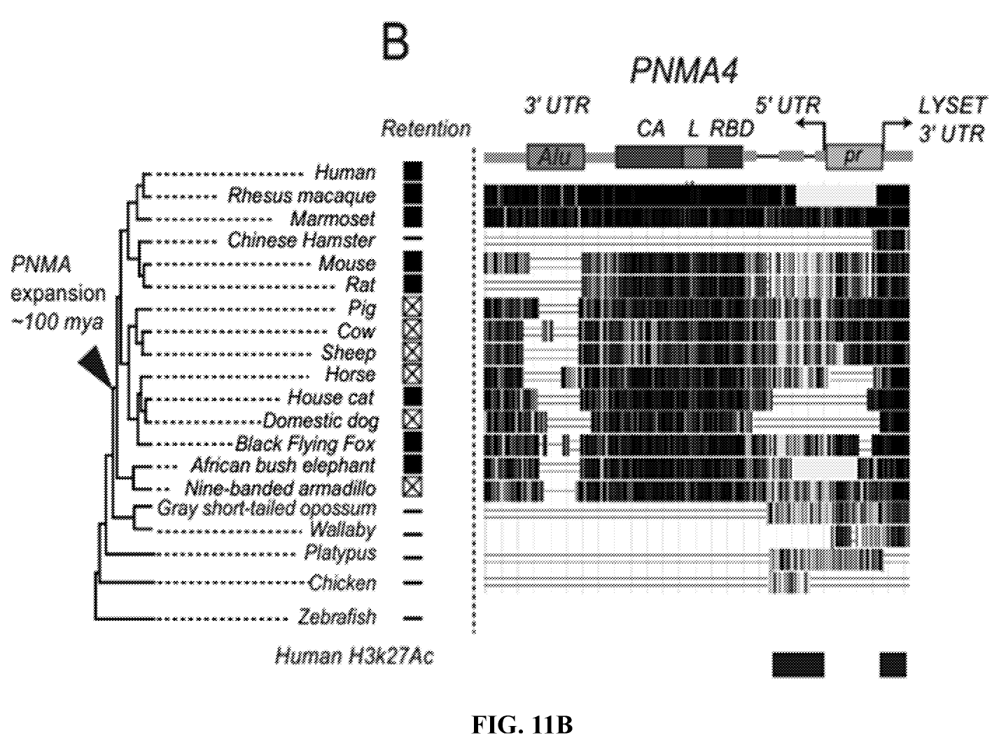

PNMA1 and PNMA4 loci are present in most (if not all) eutherians and if they serve important functions, we expect them to exhibit some degree of conservation. PNMA1 is universally conserved among eutherians and exhibits a purifying selection signature (FIG. 11A, dN/dS = 0.06)¹². The PNMA4 locus is also universal to eutherians but has undergone more diversification: some lineages have acquired a premature stop within the coding sequence leading to pseudogenization and, in primates, an Alu element has integrated in the 3’ UTR. Despite its pseudogenization in some mammals, PNMA4 also shows evidence of purifying selection (FIG. 11B, dN/dS = 0.29)¹². Consequently, two primary lines of evidence led us to investigate the possibility that these genes could be play important roles in reproductive aging: a) their expression in gonadal tissue appears to decline with age and is developmentally regulated at multiple levels and b) both show robust conservation signatures among eutherians.

### Male and female mice lacking Pnma1 or Pnma4 show premature loss of fertility

If PNMA1 and PNMA4 have been retained in mammals for reproductive functions, then we expect that animals lacking one or more of these genes should show some sort of diminution in reproductive capacity. We therefore used CRISPR-Cas9 with flanking cut sites to generate Pnma1 -/- and Pnma4 -/- mouse knockouts (KOs), and we verified that both null alleles exhibited Mendelian segregation. To validate the knockouts and to assess whether the two genes compensate for one another, we analyzed Pnma1 and Pnma4 mRNA and protein in wild type and knockouts. Rather than seeing loss of one gene leading to compensatory upregulation of the other, we observed post-transcriptional anticompensation in both knockouts: Pnma1 -/- testes showed an >80% reduction in PNMA4 protein levels (but unaltered mRNA), and vice versa for Pnma4 -/-.

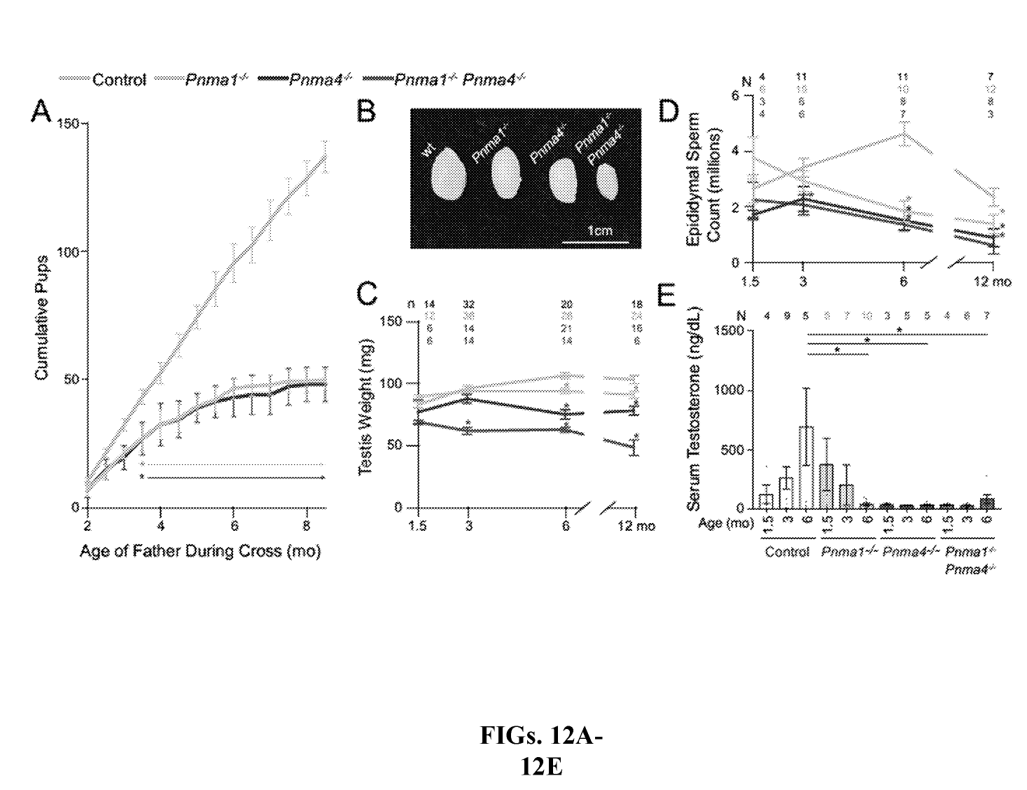

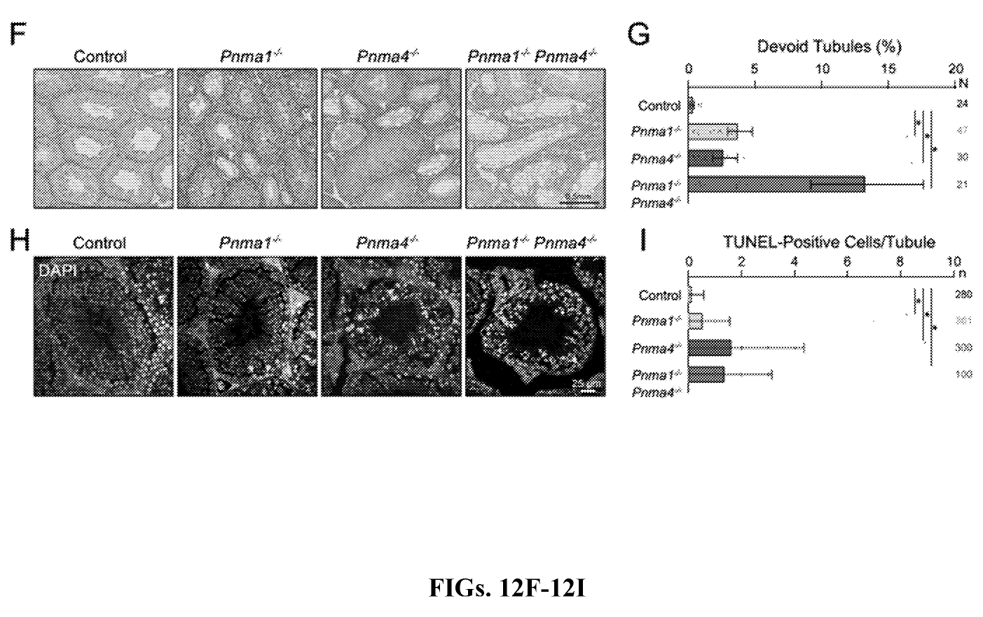

The male knockout mice did not differ from wild-type littermates at two months of age, but very soon their fertility declined, as evidenced by litter size (FIG. 3A): at three months of age, the mutants fathered litters ~70% of the size of wild-type mice, and by six months of age, their litters were only 25-50% as large as those sired by wild-type mice (FIG. 12A). There was a concomitant age-dependent testicular atrophy and decline in epididymal sperm count in single and double mutants compared to controls (FIGS. 12B-D). Serum testosterone levels in Pnma1 -/- were statistically indistinguishable from controls at 1.5 and 3 months but fell precipitously by 6 months of age (FIG. 12E). Pnma4 -/- and double knockout mice showed less than 10% of the serum testosterone levels found in control animals at any time point. Even though peritubular myoid cells (which help transport sperm) and Sertoli cells (which support the development of immature sperm) appeared normal, from 3 months onward, some seminiferous tubules in single and double mutants were devoid of undifferentiated spermatogonia, spermatocytes, and spermatids (FIGS. 12F, 12G). Because of the convoluted organization of testicular tubules, depleted areas could represent loss of developing sperm in either distinct tubules or segments of individual tubules.

To gain insight into spermatogenesis problems that ultimately lead to devoid tubules, we analyzed whether histologically normal tubules contained fewer germ cells or more apoptotic cells at 12 months. Undifferentiated spermatogonia counts, indicated by PLZF staining, did not differ from controls, but histologically normal tubules in Pnma mutants contained more apoptotic cells, as indicated by the presence of fragmented dsDNA using terminal deoxynucleotidyl transferase dUTP nick end labelling (TUNEL, FIGS. 12H, I). The depleted tubules and germ cell death support the idea that Pnma1 and Pnma4 are important for male germ cell development and likely execute their functions prior to spermatid formation.

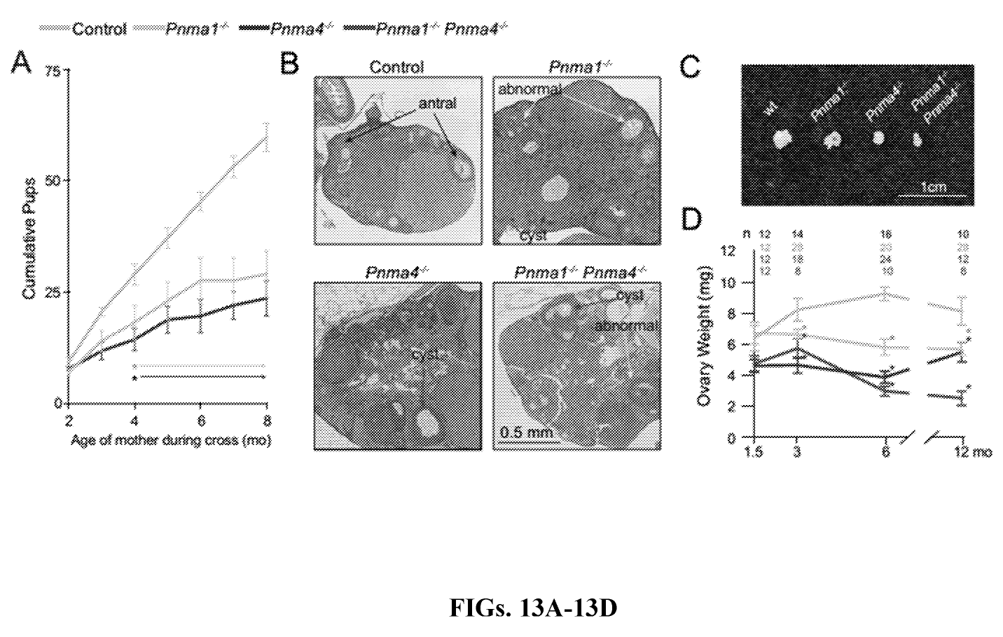

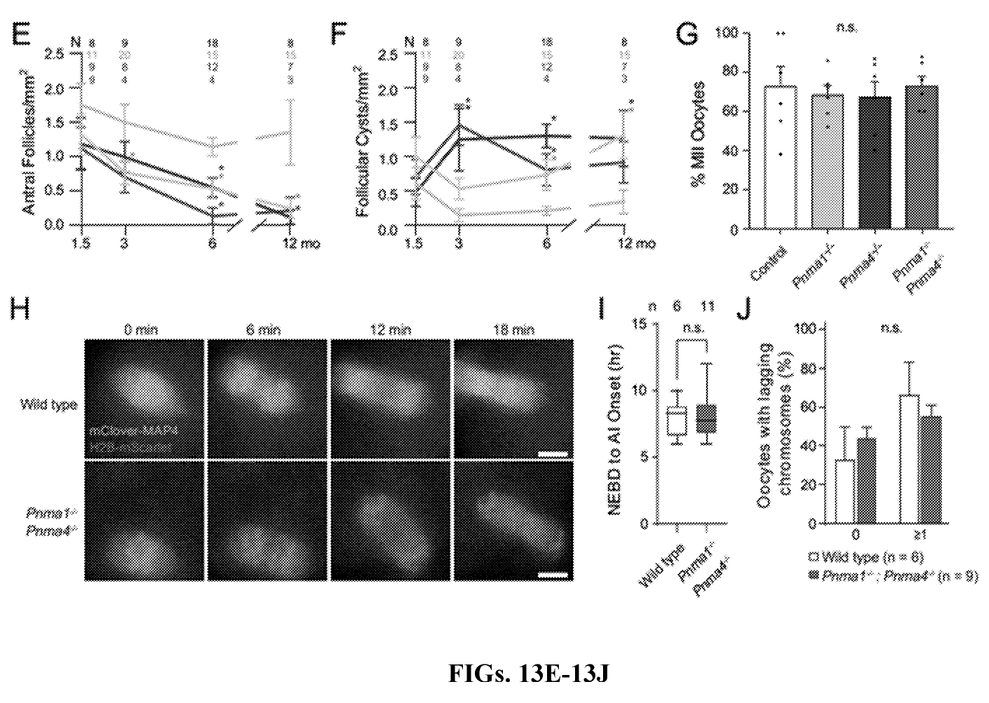

Like the male mice, female mice lacking Pnma1 or Pnma4 were similar to wild type in their reproductive capacity at two months of age, but they birthed smaller litters over time (FIG. 13A). This premature loss of fertility correlated with ovarian atrophy, diminished antral follicle counts, and a greater tendency to develop follicular cysts than wild type (FIGS. 13B-13F). Some mutant follicles had lost cumulus granulosa cells, which synthesize steroid hormones and promote oocyte growth, and resembled follicles in mice lacking estrogen receptors (ER) or follicle stimulating hormone (FSH) receptors^{22–24}. Other ovarian features did not differ significantly different between mutants and controls with some exceptions. Serum progesterone, FSH, and anti-Müllerian hormone (AMH) levels were statistically indistinguishable from controls at 1.5, 3, and 6 months with two exceptions: progesterone was down in Pnma4 -/-at 6 months and AMH was elevated in double mutants at 1.5 months. Oocytes isolated from single and double mutants completed meiosis II at 16 hours at similar frequencies compared to controls, indicating that the reduced fertility in mutant animals is unlikely to be caused by severe meiotic defects (FIG. 13G).

To determine whether mutant oocytes that do complete both meiotic divisions have subtler meiotic defects, we used high-resolution live imaging to analyze chromosome segregation during oocyte maturation. Oocytes from double mutants at two and seven months did not differ from controls in meiotic chromosome segregation, alignment on the metaphase I and metaphase II spindles, or progression timing (FIGS. 13H-3J), suggesting that mutant oocytes that do progress are meiotically competent. To assess the mouse ovarian cell types where Pnma genes may act, we analyzed the expression of Pnma1-5 in single cells at young (4.5 months), peri-estropause (10.5 months), and post-estropause (15.5 months) stages. Pnma1 and Pnma4 showed the highest expression levels throughout the ovary compared to other PNMA genes (except Pnma5 specifically in peri-estropause oocytes, orange dots). Pnma1 and Pnma4 are primarily co-expressed (by % positive cells) in granulosa, stroma, and theca cells. Orthogonal analysis of Pnma1 and Pnma4 expression from a published data set²⁵ corroborated that these genes are expressed in subpopulations of granulosa and stroma cells. In granulosa, stroma, and theca, we observed significant declines in Pnma1-positive cells at post-estropause. Pnma4 expression did not change significantly in the same time frame. Together, these data support the idea that these genes are expressed in subpopulations of ovarian cells that support oocyte maturation and viability.

### Pnma1 and Pnma4 mutants acquire abdominal obesity but appear behaviorally normal

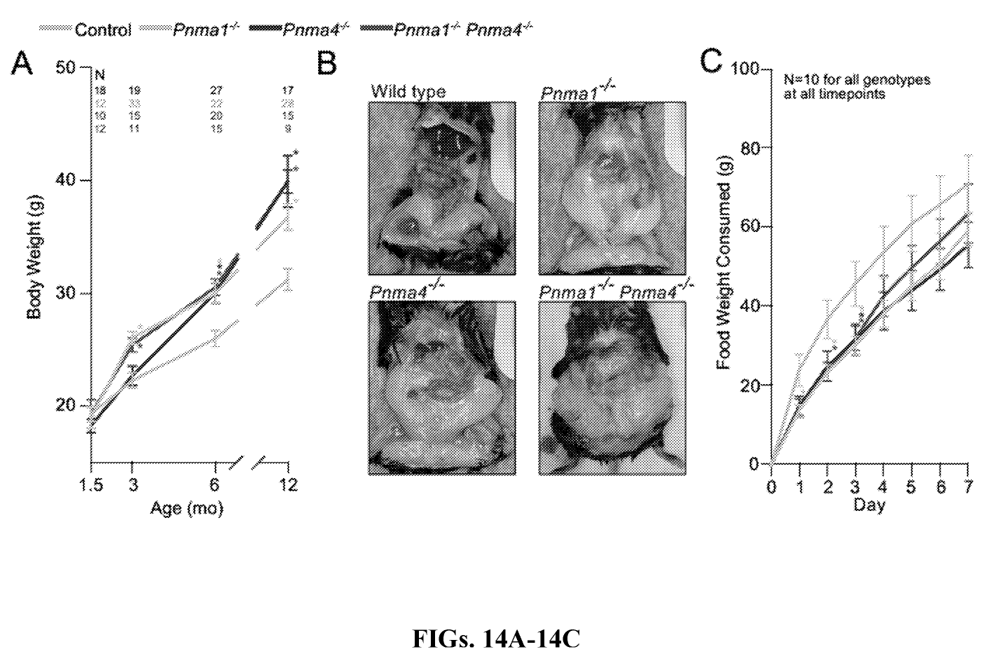

In humans and mice, reproductive decline that occurs naturally with age commonly correlates with changes in body composition^{26,27}. As male and female Pnma mutants aged, we noticed that they rapidly gained abdominal subcutaneous and visceral fat, corresponding to the time course of their dwindling fertility. At 1.5 months, the knockouts were statistically indistinguishable from controls, but by 3 months, Pnma1 -/- and double mutants were 20% heavier than controls(FIGS. 14A, 14B). By 6 months, all mutants were significantly heavier than controls. Mutant mice did not consume more food compared to controls (FIG. 14C), so the weight gain likely reflects altered metabolism. A similar phenotype is observed post-ovariectomy²⁸, in mice lacking sex hormone receptors^{22–24}, and in castrated males²⁹. In human and rodent males, increased adiposity arises due to testosterone deficiency³⁰.

Like other PNMA genes, both PNMA1 and PNMA4 are expressed in the brain³¹. We therefore tested basic neurological functions in six-month-old male Pnma1 -/-, Pnma4 -/-, and double mutants compared to wild type. The Y-maze spontaneous alternation assay revealed no deficits in short-term or spatial memory. Mutant mice were largely indistinct from controls regarding long-term memory, as assessed by the amount of post-cue freezing behavior after fear conditioning. In the open field test, mutants did not differ significantly from controls in time spent in the center, distance traveled, and vertical counts. We observed no difference in muscular strength or coordination between single and double mutants in the inverted hang or grip strength tests. Although these results revealed no obvious behavioral abnormalities in Pnma1 -/- and Pnma4 -/- male mice at 6 months of age, it is possible that female mutants or mice at other ages could display differences in cognitive function.

### PNMA1 and PNMA4 form capsid-like structures in gonadal tissue

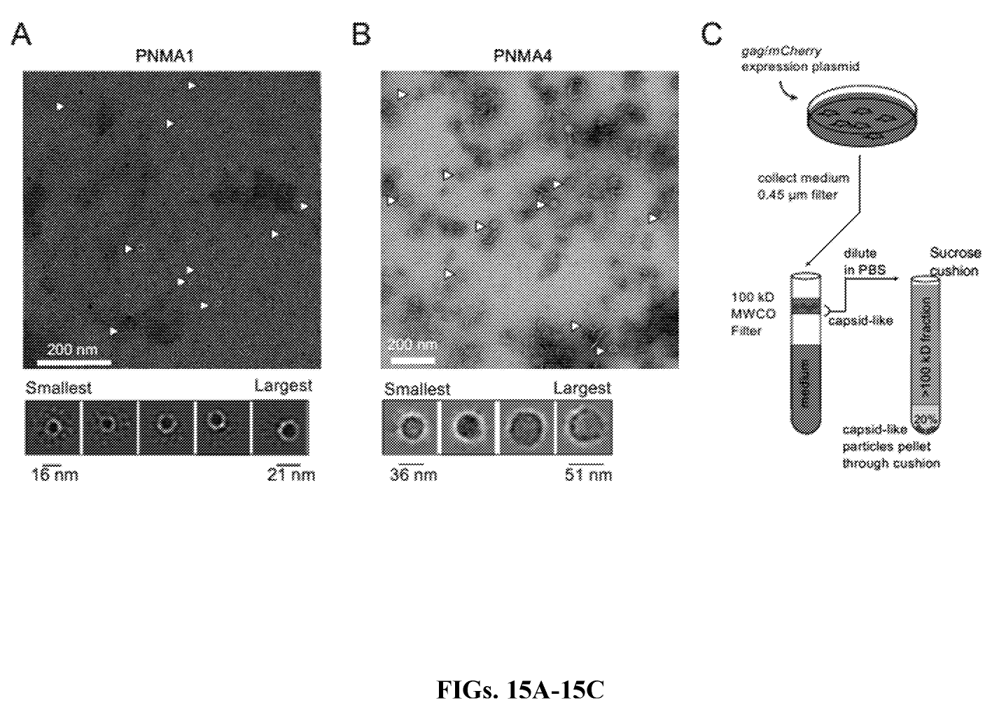

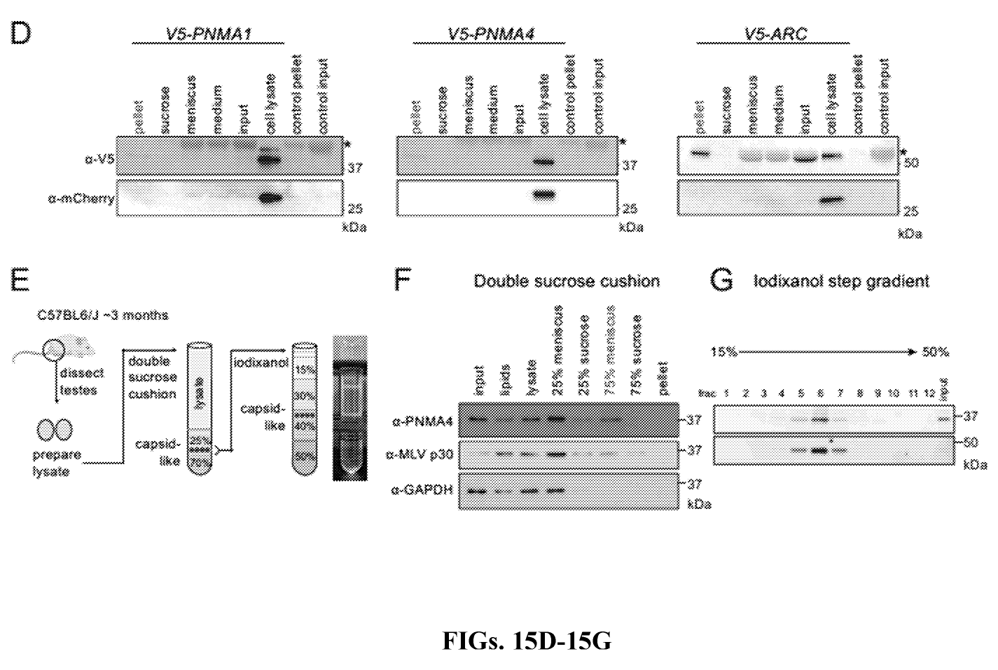

Gag family proteins self-assemble into capsid-like structures that are critical for packaging and delivering molecular cargo. One prominent example is ARC, which has been independently domesticated in mammals and Drosophila to encapsulate and traffic mRNA across neuronal synapses^{32,33}. To determine whether PNMA1 and/or PNMA4 proteins have retained the ability to self-assemble into capsids, we expressed and purified His-tagged recombinant (mouse) PNMA1 and PNMA4 from E. coli which we analyzed by size exclusion chromatography. Metaviridae capsids are amenable to isolation by size-based fractionation because they are large (yeast Ty3 capsids contain 540 gag protein subunits and are ~ 20 megadaltons³⁴). We visualized the high molecular weight fractions by negative stain transmission electron microscopy (TEM). Non-uniform capsid-like assemblies are evident in micrographs of both PNMA1 and PNMA4 (16-21 nm diameter for PNMA1 and 36-51 nm for PNMA4, FIGS. 15A, 15B). Based on their size, these results suggest that PNMA1 forms capsids similar to those formed by PNMA2³⁵ , while PNMA4 capsids are similar to those formed by LTR retroelements³⁴.

To assess capsid formation in mammalian cells, we expressed human PNMA1, PNMA4 and ARC (positive control)in HEK293T cells with mCherry (non-capsid control, driven by a separate promoter). After 24 hours, we harvested cells and fractionated cell lysate by velocity gradient ultracentrifugation. Because of their size and density, capsids migrate through the gradient, away from the major protein content of the cell. Some PNMA1 and PNMA4 appeared in denser factions. To determine if these large assemblies have the capacity to exit cells, we expressed PNMA1, PNMA4 and ARC for 24 hours in human cells, then collected the growth medium and removed the cells and any debris by filtration. We concentrated the medium using a 100 kDa molecular weight cutoff (MWCO) filter, discarded the < 100 kDa flow-through, fractionated the concentrate by sedimentation over a 20% sucrose cushion, and analyzed the fractions by immunoblot (FIGS. 15C, 15D). Like ARC, some PNMA1 and PNMA4 appeared in the pellet fraction (FIG. 15D), supporting the idea that at least some PNMA proteins exit human cells in a capsid-like form.

To assess whether PNMA proteins form capsid structures in situ, we purified PNMA4 capsids from wild-type mouse testes using a procedure that was developed for the purification of intact VLPs from plant tissue³⁶. We focused on PNMA4 because we found its detectability in testes by (polyclonal) immunoblot more reliable than PNMA1. To separate PNMA4 capsids from monomers and oligomers, we fractionated testis lysate over two sucrose cushions of 25% and 70% by ultracentrifugation (FIG. 15E). Because of their size and density, capsid particles migrate to the interface between the 25% and 70% sucrose layers and separate from monomeric proteins, which accumulate at the interface between the lysate and 25% cushion³⁶. We observed enrichment of PNMA4 at the 25%-70% interface compared to a control monomeric protein (GAPDH) and co-enrichment with p30 gag from murine leukemia virus (MLV), an enveloped capsid positive control (FIG. 15F). To separate capsids from other large structures such as ribosomes, we collected and re-fractionated the 25%-70% meniscus fraction by isopycnic (equilibrium) ultracentrifugation on an iodixanol (Optiprep) density step gradient. PNMA4 protein isolated from testis tissue co-fractionated (as large particles) with MLV p30 capsid protein (FIG. 15G). The in situ biochemical properties of PNMA4 are consistent with capsid formation, which may mediate the protein’s pro-fertility function.

### Human variation at the PNMA1 and PNMA4 loci is associated with reproductive defects

Based on the reduction of fertility in mice lacking Pnma1, Pnma4, or both, we asked whether subfertility or other reproductive phenotypes in humans are causally linked to genetic variation in the PNMA1 and/or PNMA4 loci. We used the variant-to-gene (V2G) and locus-to-gene (L2G) pipelines available from Open Targets Genetics, with a standard p-value cutoff of 5x10⁻⁸, to identify the causal gene from a trait-associated variant or locus identified by GWAS^{43,44}. Briefly, from a trait-associated variant (i.e., the polymorphism corresponding to a GWAS peak), these pipelines establish the likely causal gene by weighted analysis of independent published quantitative trait loci experiments (eQTLs, pQTLs and sQTLs), chromatin interaction experiments, in silico functional predictions, and distances between a variant and transcription start sites. We found six variants attributed to PNMA1 (from nine studies and three publications^{45–47}) associated with altered testosterone levels and levels of sex hormone binding globulin (SHBG, levels correlate to testosterone). Two variants attributed to PNMA4 are associated with altered age of menarche⁴⁸ and male puberty timing⁴⁹. We also identified two additional variants associated with elevated body fat (thirteen studies, three publications^{50–52}) that are attributed to PNMA4. These results support the notion that the alterations in reproductive capacity we observed in mouse Pnma1 and Pnma4 knockouts are relevant to humans.

### Discussion

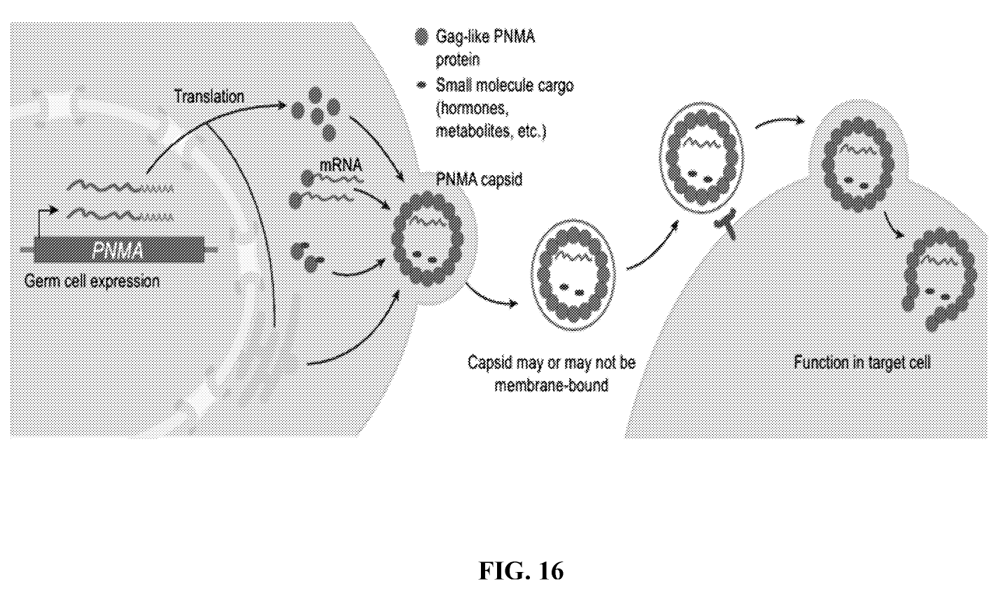

In general, retroelement gag capsids facilitate endogenous and parasitic intercellular communication. Like many other tissues, gonads rely on endocrine, paracrine, and autocrine signaling to support gamete development, and their cells employ a variety of means to communicate with each other. For example, testosterone, produced largely in Leydig cells, diffuses into the testicular tubules where it binds to androgen receptors on Sertoli cells which then drive the production of factors that support spermatogenesis^{53,54}. Progesterone, produced by follicular granulosa cells and corpora lutea, is critical for successful oocyte maturation and ovulation^{55,56}. Communication among germ cells also takes place through intercellular bridges that connect the cytoplasm of adjacent cells into a syncytium; males that cannot form these bridges (such as Tex14 knockouts) are sterile⁵⁷ (these structures are important, but not essential, for female fertility^{58,59}). These germ cell bridges/syncytia have been proposed to foster dosage compensation in haploid cells, coordination of meiotic entry, and sharing of signals for synchronous cell divisions within seminiferous tubules^{57,58,60}. In the ovary, oocytes are surrounded by cumulus/coronal granulosa cells that extend long processes termed transzonal projections through the zona pellucida^{61–63}. These projections release vesicles that are thought to contain RNA and have been proposed to enable sharing of RNA between transcriptionally active granulosa cells and the silent oocyte^{64,65}. Conversely, oocytes release growth factors that regulate proliferation of granulosa cells. Emerging evidence supports the notion that communication between follicular cells and oocytes is important for maintaining germ cell fitness: aged oocytes transplanted into young follicles become rejuvenated, improving in mitochondrial function and the fidelity of meiotic chromosome segregation, among other measures⁶⁶. Although PNMA1 and PNMA4 are not essential for intercellular communication (the double mutant would be sterile), we propose that PNMA1 and PNMA4 capsids could promote the transmission of cytoplasmic signals, whether through bridges, transzonal projections, and/or by transport across plasma membranes (FIG. 16).

The word capsid derives from the Latin word for “case,” and capsids form an elegant means to carry molecular messages. The question we still need to answer is, do the capsids formed by PNMA1 and PNMA4 carry cargo, and if so, what is it? This cargo could conceivably be nucleic acids such as mRNA or tRNA, hormones such as testosterone or FSH, or a metabolite—but whatever the cargo, it likely has important functions for fertility.

PNMA4 has been implicated in apoptosis in the context of mis-expression in cancer cells^{67–69}. PNMA4 interacts with the pro-apoptotic protein Bax and the pro-survival proteins Bcl-2 and Bcl-X(L) and can mediate caspase-dependent apoptosis when overexpressed⁷⁰. Given that 12-month-old Pnma4 ^{-/-}testes exhibit greater numbers of TUNEL-positive cells, PNMA4 likely promotes survival in gonadal tissue. In mutant males, we observed an age-dependent intra-organ heterogeneity where some testicular tubule segments were devoid of sperm and sperm precursor cells while others appeared wild type. Other than increased cell death in aged tubules, we did not observe any clear defects in the ‘normal’-appearing seminiferous tubules of mutant animals. Because mutant tubule segments are either devoid or full, without an obvious intermediate state, we speculate that devoid tubule segments have experienced a failure to initiate a wave of spermatogenesis and, once a failure has occurred, that tubule cannot recover.

Female mice lacking Pnma1 and/or Pnma4 exhibit a wide range of ovarian abnormalities. Bmp6 -/- , Bmp15 -/- , Smad2/3^{cko} , and Smad3^{cko} female mice exhibit age-dependent subfertility with ovarian histology reminiscent of Pnma knockouts^{71–74}, suggesting that the Pnma1 and Pnma4 mutant phenotypes could, in part, be due to downregulated TGF-beta signaling. In mutant females, the oocytes that are produced appear healthy and undergo meiosis. Similarly, while sperm count declines with age in mutant males, the sperm within the epididymis appear normal. These data are consistent with a model in which the function(s) of PNMA1 and PNMA4 are enacted prior to gamete release from the ovary/testis though we cannot rule out defects in later processes such as implantation.

Developing yeast gametes thwart proliferation of Ty3, a parasitic ancestor of PNMA genes, by using amyloid-like condensates of the RNA-binding protein Rim4, which binds retrotransposon mRNA and inhibits its translation¹⁰. Mammalian analogs of Rim4 include DAZ, DAZL (DAZ-like), and PUM1/2 (Pumilio). Although higher-order assemblies of RNA-binding proteins behave somewhat differently in yeast and mammalian germ cells, DAZ, DAZL, and PUM1/2 are like Rim4 in that they regulate translation in developing gametes, affect gene expression post-transcriptionally by binding the UTRs of their target genes, and form condensates in vivo ^{75–78}. Our analysis of DAZL-mediated regulation of PNMA genes supports the idea that, from yeast to humans, RNA-binding proteins with the propensity to form condensates can regulate the translation of retroelement-derived mRNA. We propose that as Metaviridae elements gradually evolved beneficial functions and lost proliferative capacity in host meiotic cells, the analogs of condensates acting as a defense mechanism (Rim4) were repurposed in mammals as regulators (DAZL). It is possible that DAZL have maintained a role to protect sperm and eggs from the assault of expressed Metaviridae retrotransposons. Several human endogenous retroviruses (hERVs) are robustly expressed in gonads^{79,80} and these elements do not appear to be rapidly proliferating, suggesting that the hERV lifecycle is somehow blocked after mRNA transcription. The possibility for condensate-forming RNA-binding proteins to post-transcriptionally regulate hERVs has precedent: in neurons, the neurodegeneration-associated protein TDP-43 binds to hERV-K mRNA and regulates its protein synthesis⁸¹.

### REFERENCES

1. Graaff, A. A. de, Land, J. A., Kessels, A. G. H. & Evers, J. L. H. Demographic age shift toward later conception results in an increased age in the subfertile population and an increased demand for medical care. Fertil Steril 95, 61–63 (2011).
2. Petraglia, F., Serour, G. I. & Chapron, C. The changing prevalence of infertility. Int J Gynecol Obstet 123, S4–S8 (2013).
3. Laureau, R. et al. Meiotic Cells Counteract Programmed Retrotransposon Activation via RNA-Binding Translational Repressor Assemblies. Developmental cell 56, 22-35.e7 (2021).
4. Campillos, M., Doerks, T., Shah, P. K. & Bork, P. Computational characterization of multiple Gag-like human proteins. Trends in genetics : TIG 22, 585–589 (2006).
5. Kearney, M. S., Levine, P. B. & Pardue, L. The Puzzle of Falling US Birth Rates since the Great Recession. J Econ Perspect 36, 151–176 (2022).
6. Cordaux, R. & Batzer, M. A. The impact of retrotransposons on human genome evolution. Nature Reviews Genetics 10, 691–703 (2009).
7. Branciforte, D. & Martin, S. L. Developmental and Cell Type Specificity of LINE-1 Expression in Mouse Testis: Implications for Transposition. Mol Cell Biol 14, 2584–2592 (1994).
8. Kojima, K. K. Human transposable elements in Repbase: genomic footprints from fish to humans. Mobile DNA 9, 2–14 (2018).
9. Gnoth, C. et al. Definition and prevalence of subfertility and infertility. Hum Reprod 20, 1144–1147 (2005).
10. Gurunath, S., Pandian, Z., Anderson, R. A. & Bhattacharya, S. Defining infertility—a systematic review of prevalence studies. Hum Reprod Update 17, 575–588 (2011).
11. Matzuk, M. M. & Lamb, D. J. The biology of infertility: research advances and clinical challenges. Nat Med 14, 1197–1213 (2008).
12. Wong, W. Y., Thomas, C. M. G., Merkus, J. M. W. M., Zielhuis, G. A. & Steegers-Theunissen, R. P. M. Male factor subfertility: possible causes and the impact of nutritional factors. Fertil Steril 73, 435–442 (2000).
13. Delbaere, I., Verbiest, S. & Tydén, T. Knowledge about the impact of age on fertility: a brief review. Upsala J Med Sci 125, 1–8 (2020).
14. Harris, I. D., Fronczak, C., Roth, L. & Meacham, R. B. Fertility and the aging male. Rev Urology 13, e184-90 (2008).
15. Munama, M., Md., A. T. C., Nasima, B. & Sayeda, R. Types of Infertility and Its Risk Factors among Infertile Women: A Prospective Study in Dhaka City. J Biosci Medicines 10, 158–168 (2022).
16. Zorrilla, M. & Yatsenko, A. N. The Genetics of Infertility: Current Status of the Field. Curr Genetic Medicine Reports 1, 247–260 (2013).
17. Zhang, X.-L. et al. Pnma5 is essential to the progression of meiosis in mouse oocytes through a chain of phosphorylation. Oncotarget 8, 96809–96825 (2017).
18. Schüller, M., Jenne, D. & Voltz, R. The human PNMA family: novel neuronal proteins implicated in paraneoplastic neurological disease. Journal of neuroimmunology 169, 172–176 (2005).
19. Mi, S. et al. Syncytin is a captive retroviral envelope protein involved in human placental morphogenesis. Nature 403, 785–789 (2000).
20. Ono, R. et al. Deletion of Peg10, an imprinted gene acquired from a retrotransposon, causes early embryonic lethality. Nature genetics 38, 101–106 (2006).
21. Sekita, Y. et al. Role of retrotransposon-derived imprinted gene, Rtl1, in the feto-maternal interface of mouse placenta. Nature genetics 40, 243–248 (2008).
22. Tan, K. O. et al. MAP-1, a novel proapoptotic protein containing a BH3-like motif that associates with Bax through its Bcl-2 homology domains. The Journal of biological chemistry 276, 2802–2807 (2001).
23. Nikolaienko, O., Patil, S., Eriksen, M. S. & Bramham, C. R. Arc protein: a flexible hub for synaptic plasticity and cognition. Semin Cell Dev Biol 77, 33–42 (2018).
24. Segel, M. et al. Mammalian retrovirus-like protein PEG10 packages its own mRNA and can be pseudotyped for mRNA delivery. Sci New York N Y 373, 882–889 (2021).
25. Garfinkel, D. J., Boeke, J. D. & Fink, G. R. Ty element transposition: reverse transcriptase and virus-like particles. Cell 42, 507–517 (1985).
26. Wicker, T. et al. A unified classification system for eukaryotic transposable elements. Nature Reviews Genetics 8, 973–982 (2007).
27. Miga, K. H. et al. Telomere-to-telomere assembly of a complete human X chromosome. Nature 585, 79–84 (2020).
28. Soumillon, M. et al. Cellular source and mechanisms of high transcriptome complexity in the mammalian testis. Cell reports 3, 2179–2190 (2013).
29. Dalmau, J. et al. Ma1, a novel neuron- and testis-specific protein, is recognized by the serum of patients with paraneoplastic neurological disorders. Brain : a journal of neurology 122 ( Pt 1), 27–39 (1999).
30. Voltz, R. et al. A serologic marker of paraneoplastic limbic and brain-stem encephalitis in patients with testicular cancer. The New England journal of medicine 340, 1788–1795 (1999).
31. Rosenfeld, M. R., Eichen, J. G., Wade, D. F., Posner, J. B. & Dalmau, J. Molecular and clinical diversity in paraneoplastic immunity to Ma proteins. Annals of Neurology 50, 339–348 (2001).
32. Anderson, E. L. et al. Stra8 and its inducer, retinoic acid, regulate meiotic initiation in both spermatogenesis and oogenesis in mice. Proceedings of the National Academy of Sciences 105, 14976–14980 (2008).
33. Kojima, M. L., Rooij, D. G. de & Page, D. C. Amplification of a broad transcriptional program by a common factor triggers the meiotic cell cycle in mice. eLife 8, 720 (2019).
34. Bolcun-Filas, E. et al. A-MYB (MYBL1) transcription factor is a master regulator of male meiosis. Development 138, 3319–3330 (2011).
35. Zhou, L. et al. BTBD18 Regulates a Subset of piRNA-Generating Loci through Transcription Elongation in Mice. Developmental cell 40, 453-466.e5 (2017).
36. Kokošar, J. & Kordiš, D. Genesis and regulatory wiring of retroelement-derived domesticated genes: a phylogenomic perspective. Molecular biology and evolution 30, 1015–1031 (2013).
37. Lesch, B. J., Silber, S. J., McCarrey, J. R. & Page, D. C. Parallel evolution of male germline epigenetic poising and somatic development in animals. Nature genetics 48, 888–894 (2016).
38. Marin, R. et al. Convergent origination of a Drosophila-like dosage compensation mechanism in a reptile lineage. Genome research 27, 1974–1987 (2017).
39. Chen, J. et al. Genome-wide analysis of translation reveals a critical role for deleted in azoospermia-like (Dazl) at the oocyte-to-zygote transition. Genes & Development 25, 755–766 (2011).
40. Collier, B., Gorgoni, B., Loveridge, C., Cooke, H. J. & Gray, N. K. The DAZL family proteins are PABP-binding proteins that regulate translation in germ cells. The EMBO Journal 24, 2656–2666 (2005).
41. Yang, C.-R. et al. The RNA-binding protein DAZL functions as repressor and activator of mRNA translation during oocyte maturation. Nature Communications 11, 1399–16 (2020).
42. Berchowitz, L. E. et al. Regulated Formation of an Amyloid-like Translational Repressor Governs Gametogenesis. Cell 163, 406–418 (2015).
43. Rosario, R., Smith, R. W. P., Adams, I. R. & Anderson, R. A. RNA immunoprecipitation identifies novel targets of DAZL in human foetal ovary. Molecular human reproduction 23, 177–186 (2017).
44. Zagore, L. L. et al. DAZL Regulates Germ Cell Survival through a Network of PolyA-Proximal mRNA Interactions. Cell reports 25, 1225-1240.e6 (2018).
45. Tan, C. T. et al. MOAP-1 Mediates Fas-Induced Apoptosis in Liver by Facilitating tBid Recruitment to Mitochondria. Cell reports 16, 174–185 (2016).
46. Boekhout, M. et al. REC114 Partner ANKRD31 Controls Number, Timing, and Location of Meiotic DNA Breaks. Mol Cell 74, 1053-1068.e8 (2019).
47. Schomberg, D. W. et al. Targeted Disruption of the Estrogen Receptor-α Gene in Female Mice: Characterization of Ovarian Responses and Phenotype in the Adult. Endocrinology 140, 2733–2744 (1999).
48. Danilovich, N. et al. Estrogen deficiency, obesity, and skeletal abnormalities in follicle-stimulating hormone receptor knockout (FORKO) female mice. Endocrinology 141, 4295–308 (2000).
49. Ke, H. Z. et al. Effects of CP-336,156, a New, Nonsteroidal Estrogen Agonist/Antagonist, on Bone, Serum Cholesterol, Uterus, and Body Composition in Rat Models. Endocrinology 139, 2068–2076 (1998).
50. Heine, P. A., Taylor, J. A., Iwamoto, G. A., Lubahn, D. B. & Cooke, P. S. Increased adipose tissue in male and female estrogen receptor-α knockout mice. Proc National Acad Sci 97, 12729–12734 (2000).
51. Ahmed, E. A. & Rooij, D. G. de. Staging of mouse seminiferous tubule cross-sections. Methods Mol Biology Clifton N J 558, 263–77 (2009).
52. KANATSU-SHINOHARA, M., CHEN, G., MORIMOTO, H. & SHINOHARA, T. CD2 is a surface marker for mouse and rat spermatogonial stem cells. J Reproduction Dev 66, 341–349 (2020).
53. Kopylow, K. von & Spiess, A.-N. Human spermatogonial markers. Stem Cell Res 25, 300–309 (2016).
54. Lammers, J. H. et al. The gene encoding a major component of the lateral elements of synaptonemal complexes of the rat is related to X-linked lymphocyte-regulated genes. Molecular and Cellular Biology 14, 1137–1146 (1994).
55. Zickler, D. & Kleckner, N. Meiotic chromosomes: integrating structure and function. Annual review of genetics 33, 603–754 (1999).
56. Mahadevaiah, S. K. et al. Recombinational DNA double-strand breaks in mice precede synapsis. Nature genetics 27, 271–276 (2001).
57. Tarsounas, M., Morita, T., Pearlman, R. E. & Moens, P. B. RAD51 and DMC1 form mixed complexes associated with mouse meiotic chromosome cores and synaptonemal complexes. J Cell Biol 147, 207–220 (1999).
58. Gray, S. & Cohen, P. E. Control of Meiotic Crossovers: From Double-Strand Break Formation to Designation. Annu Rev Genet 50, 175–210 (2016).
59. Hunter, N. Meiotic Recombination: The Essence of Heredity. Csh Perspect Biol 7, a016618 (2015).
60. Royo, H. et al. Evidence that Meiotic Sex Chromosome Inactivation Is Essential for Male Fertility. Curr Biol 20, 2117–2123 (2010).
61. Bolcun-Filas, E. & Handel, M. A. Meiosis: the chromosomal foundation of reproduction. Biol Reprod 99, 112–126 (2018).
62. Turner, J. M. A. Meiotic Silencing in Mammals. Annu Rev Genet 49, 395–412 (2015).
63. Yu, J., Hecht, N. B. & Schultz, R. M. Expression of MSY2 in Mouse Oocytes and Preimplantation Embryos1. Biol Reprod 65, 1260–1270 (2001).
64. Lee, S. S. et al. TRIM39 is a MOAP-1-binding protein that stabilizes MOAP-1 through inhibition of its poly-ubiquitination process. Experimental cell research 315, 1313–1325 (2009).
65. Clift, D., So, C., McEwan, W. A., James, L. C. & Schuh, M. Acute and rapid degradation of endogenous proteins by Trim-Away. Nat Protoc 13, 2149–2175 (2018).
66. Pastuzyn, E. D. et al. The Neuronal Gene Arc Encodes a Repurposed Retrotransposon Gag Protein that Mediates Intercellular RNA Transfer. Cell 173, 275 (2018).
67. Vos, M. D. et al. The RASSF1A Tumor Suppressor Activates Bax via MOAP-1. J Biol Chem 281, 4557–4563 (2006).
68. Fu, N. Y., Sukumaran, S. K. & Yu, V. C. Inhibition of ubiquitin-mediated degradation of MOAP-1 by apoptotic stimuli promotes Bax function in mitochondria. P Natl Acad Sci Usa 104, 10051–6 (2007).
69. Kurilo, L. F. Oogenesis in antenatal development in man. Human genetics 57, 86–92 (1981).
70. Malki, S., Heijden, G. W. van der, O’Donnell, K. A., Martin, S. L. & Bortvin, A. A role for retrotransposon LINE-1 in fetal oocyte attrition in mice. Developmental cell 29, 521–533 (2014).
71. Licatalosi, D. D. et al. HITS-CLIP yields genome-wide insights into brain alternative RNA processing. Nature 456, 464–469 (2008).
72. Tan, C. T. et al. MOAP‐1‐mediated dissociation of p62/SQSTM1 bodies releases Keap1 and suppresses Nrf2 signaling. Embo Rep 22, e50854 (2021).
73. Ashley, J. et al. Retrovirus-like Gag Protein Arc1 Binds RNA and Traffics across Synaptic Boutons. Cell 172, 262-274.e11 (2018).
74. Xu, J. et al. PNMA2 forms non-enveloped virus-like capsids that trigger paraneoplastic neurological syndrome. Biorxiv Prepr Serv Biology 2023.02.09.527862 (2023) doi:10.1101/2023.02.09.527862.
75. Dodonova, S. O., Prinz, S., Bilanchone, V., Sandmeyer, S. & Briggs, J. A. G. Structure of the Ty3/Gypsy retrotransposon capsid and the evolution of retroviruses. Proc National Acad Sci 116, 10048–10057 (2019).
76. Mastronarde, D. N. Automated electron microscope tomography using robust prediction of specimen movements. J Struct Biol 152, 36–51 (2005).
77. Punjani, A., Rubinstein, J. L., Fleet, D. J. & Brubaker, M. A. cryoSPARC: algorithms for rapid unsupervised cryo-EM structure determination. Nat Methods 14, 290–296 (2017).

What is claimed is:

1. A method for improving fertility or treating infertility in a subject comprising administering to the subject an amount of capsids comprising PNMA1 and/or PNMA4 effective to improve fertility or treat infertility in a subject.

2. The method of claim 1, wherein the capsids have been previously obtained from a mammalian subject.

3. The method of claim 1, wherein the capsids have been previously obtained mammalian testis or ovary tissues.

4. The method of claim 1, wherein the PNMA1 and/or PNMA4-comprising capsids are obtained from cultured cells bioengineered to express PNMA1 and/or PNMA4.

5. The method of claim 1, wherein the capsids are endogenous capsids.

6. The method of claim 1, wherein the capsids have not been genetically engineered.

7. The method of claim 1, wherein the capsids have not been altered to contain RNA.

8. The method of claim 1, wherein the capsids are administered directly to the testes of a male subject or to the ovary of a female subject.

9. The method of claim 1, wherein the capsids have been purified from a biological sample by size and/or by density.

10. The method of claim 1, wherein the capsids have been purified from a biological sample via antibody purification.

11. The method of claim 1, wherein the capsids have been purified from a biological sample which is a reproductive tissue.

12. The method of claim 1, wherein the capsids are human.

13. The method of claim 1, wherein the subject has an age-related fertility decline or sub-fertility.

14. The method of claim 1, wherein the capsids comprise PNMA4 but not PNMA1.

15. The method of claim 1, wherein the subject is a human.

16. A pharmaceutical composition comprising an amount of purified capsids comprising PNMA1 and/or PNMA4 and a pharmaceutically acceptable carrier.

17. The pharmaceutical composition of claim 16, wherein the capsids comprise PNMA4 but not PNMA1.

18. The pharmaceutical composition of claim 17, wherein the capsids have been previously obtained mammalian testis or ovary tissues.

19. A method of making a pharmaceutical composition for treating infertility or subfertility in a subject comprising isolating from a biological sample capsids comprising PNMA1 and/or PNMA4 and admixing the capsids with a pharmaceutically acceptable carrier.

20. The method of claim 19, further comprising purifying the capsids comprising PNMA1 and/or PNMA4 prior to admixing the capsids with a pharmaceutically acceptable carrier.
